(19) United States
(12) Patent Application Publication
(10) Pub. No.: US 2009/0004668 A1
(43) Pub. Date: Jan. 1, 2009

(54) Pre-miRNA loop-modulated target regulation

(75) Inventors: Chang-Zheng Chen, Stanford, CA (US); Hyeyoung Min, Seoul (KR); Gwen Liu, Newport Coast, CA (US)

Correspondence Address: ROBINS & PASTERNAK, 1731 EMBARCADERO ROAD, SUITE 230, PALO ALTO, CA, 94303, US

(73) Assignee: The Board of Trustees of the Leland Stanford Junior University

(21) Appl. No.: 12/214,793

(22) Filed: Jun. 20, 2008

Related U.S. Application Data
(60) Provisional application No. 60/936,869, filed on Jun. 22, 2007.

(51) Int. Cl. C12Q 1/68; C07H 21/02; C07H 21/04; C12N 5/06

(52) U.S. Cl. 435/6; 536/23.1; 435/375; 536/24.1

(57) ABSTRACT

By employing essential nucleotides from both the stem and loop of precursor-miRNA, greater specificity is achieved as to the mRNAs that are repressed. It is found that besides the seed sequence of the stem of the pre-miRNA, nucleotides in the loop affect the activity and specificity of the cursor- and the processing and binding to target mRNA. By using both sequences in the natural pre-miRNA or modified mimetics, one can screen for cellular miRNA expression, modulate cell properties with greater specificity and investigate cellular activity as to phenotype and response to external stimuli in the presence and absence of target protein expression.

### CROSS-REFERENCE TO RELATED APPLICATIONS

This application claims benefit under 35 U.S.C. § 119(e)(1) of provisional application 60/936,869 filed Jun. 22, 2007, which application is hereby incorporated by reference in its entirety.

### GOVERNMENT SUPPORT

This work was supported by NIH R01-HL081612 grant and the Baxter and Terman faculty awards (to C.-Z. C.). The U.S. government may have an interest in this invention.

### INTRODUCTION

1. Technical Field

The field of this invention is the preparation and use of pre-miRNA molecules with distinct loop nucleotides for quantitative and selective control of the expression of mRNA targets.

2. Background

One of the most important developments in the 21^{st} century is the use of RNAi to modulate expression in cells. Use of RNAi avoids the tedious use of knockout protocols, while allowing for the determination of the effect of reduced or eliminated expression of a protein on the phenotype of a cell. The major variations of RNAi are referred to as miRNA (microRNA), siRNA (short interfering RNA) and piRNA (piwi RNA).

For miRNA the conventional wisdom is that a gene encodes the miRNA in a form referred to as the primary-miRNA (pri-miRNA). The gene may be in any portion of the genome, frequently being found in regions that do not code for proteins and in introns. Not infrequently, a number of pri-miRNA genes are found in proximity, where the mature miRNAs will differ by only a few nucleotides, providing a group of isoforms that appear to have similar binding specificities and affinities. The expressed pri-miRNA will generally contain from a few hundred to a few thousand nucleotides. The pri-miRNA is then processed in the nucleus by the proteins Drosha and Pasha to the pre-miRNA that has a stem and loop structure with flanking sequences. The pre-miRNA will generally have about 60 bases to 70 bases. The pre-miRNA is then actively transported into the cytoplasm by exportin 5 and Ran-GTP. In the cytoplasm, the pre-miRNA is then further processed into small RNA duplexes of approximately 22 nucleotides by the proteins Dicer and Loquacious. The functional or guiding strand of the miRNA duplex is then loaded into the RNA-induced silencing complex (RISC). Finally, the miRNA guiding strand guides the RISC to the cognate messenger RNA (mRNA) target for translational repression or degradation of the mRNA.

The miRNA is frequently found to lack perfect complementarity with the target mRNA. Frequently, there are bulges, e.g. mismatches, deletions and insertions, not only between the target mRNA and the mature miRNA, but also between the two chains of the stem of the pre-miRNA. Also, it has frequently been found that more than one mRNA may be regulated by the same mature miRNA. A sequence of the miRNA from 5′-nucleotides 1-10. usually 1-8, is called the “seed” sequence. A sequence of from 7-8 nucleotides is found sufficient to recognize and bind to the target mRNA and provide translational repression or mRNA destabilization, while fewer nucleotides may still provide repression where there is substantial complementarity between the 3′ miRNA sequence and a target mRNA sequence in proximity to the sequence binding to the 5′ miRNA sequence.

There have been extensive efforts to define the sequences in the target mRNAs and the miRNAs that define effective binding between the two RNA species. It is frequently found that there are numerous mRNAs complementary to the same seed sequences, so that the miRNA has a potentially large repertoire for regulation. It remains a conundrum how the miRNA provides for specific regulation in light of the frequency of seed sequences and the substantial redundancy of seed sequences among miRNAs.

Because of the evident importance of miRNA in cell regulation—miRNAs have been found to be associated with cancers and other diseases—there is great interest in understanding the mechanism whereby the miRNA regulates expression. In addition, the repertoire of miRNAs being expressed in a cell has been found to be associated with various indications and may indicate the severity of the indication and potentially a particular therapeutic protocol. Also, there is a great effort to develop miRNAs that may have therapeutic activity for the treatment of various diseases, such as cancer, where down regulation of one or more genes may inhibit tumor growth, control autoimmune diseases, correct genetic deficiencies associated with the expression of miRNAs and the like. In order to prepare arrays for screening miRNA profiles of cells it will be necessary to better understand the binding requirements between the miRNA and the target(s) mRNAs. To prepare drugs that have specificity for one or a few mRNAs, it will be essential to be able to design miRNAs that are specific for the desired targets and/or have substantially attenuated activity toward mRNAs other than the target mRNA. It will also be critical to be able to design antisense oligonuclotides (RNA and DNA) that can specifically silence miRNA genes that encode identical or nearly identical mature miRNAs. It is therefore of great interest to find additional components of the miRNA regulation that will allow improved identification of miRNAs and their targets, enhanced specificity for the miRNA toward the desired target and discrimination between the activities of miRNAs sharing substantial homology in the seed region.

### Literature

Reviews of miRNA may be found in Ying, et al 2006 Methods Mol Biol 342, 1-18 and Dykxhom (2006) Gene Therapy, 1-12. References concerned with miRNA including applications for miRNA with translation suppression include: Qi-Jing Li, et al 2007 Cell 129, 147-61 (hematopoiesis regulation); Chang-Zheng 2005 NEJM 353, 1768-71 (oncogene and tumor suppression); Beuvink, et al. 2007 Nucl Acids Res 35:e52 (miRNA array); Lin, et al 2006 Methods Mol Biol 342, 313-29 (intronic miRNA); Nilsen 2007 Trends in Genetics 23:243-9; Gaidatzis, et al 2007 8, 69 (predicting targets); Schmittgen 2004 Nucleic Acids Res 32:e43 (PCR amplification of genomic miRNA); Krutzfeldt, et al 2007 Nucleic Acids Res 35, 2885-92; and Li, et al 2007 Cell 129, 147-61 (“antagomirs” modified miRNA). Patent references of interest, particularly for the preparation of arrays and selection of probes include: U.S. patent applications 2007/0099196 (probes using LNA); 2007/0100563 (probe selection methods); 2007/0099193 (modified probes); 2007/0009915 (miRNA identification with DNA and nuclease); 2006/0130176 (modifies miRNA to enhance complementarity); 2005/0277139 (miRNA array); 2005/0075492 (preparation and use of miRNA) and 2004/0053411 (miRNA genomic precursor construct for translation suppression). See also, U.S. Pat. Nos. 7,056,704 and 7,078,196 (preparation of miRNA molecules). Synthetic miRNAs are described in Vatolin, et al 2006 J Mol Biol 358, 983-6 and Tsuda, et al 2005 Int J Oncol 27, 1299-306. These references are incorporated herein in their entirety as if completely set forth herein.

### SUMMARY OF THE INVENTION

In accordance with the subject invention precursors for mature miRNAs (precursor miRNAs), including pre-miRNAs and pri-miRNAs, are employed to provide a more selective range of mRNA targets than are normally obtained with mature miRNAs. The naturally occurring precursor miRNA molecules may be modified for increasing selectivity in binding to target mRNA or synthetic precursor miRNAs prepared differing from the wild-type pre-miRNAs portion in their flanking region, stem and/or loop. Either the stem or loop regions may be modified, where the loop regions are identified as enhancing selectivity for target mRNAs, whereby target mRNAs are determined based on complementarity to both the guiding sequence of the stem and at least 3 nt of the loop. Alternatively, synthetic pre-miRNAs and pri-miRNAs are prepared where the seed sequence and at least 2 nt of the loop are complementary to the target mRNA, desirably without bulges or interruptions between binding nucleotides.

In developing algorithms for determining target mRNAs from mature miRNA, or synthetic miRNAs for target mRNA, the algorithms are modified to include complementarity to at least a sequence of 2 nts of the loop sequence, desirably in a portion of the loop in proximity to the seed sequence. Precursor miRNAs are screened for their binding profile of mRNAs in a mixture of mRNAs in vitro and in vivo and specific targets are identified by stem and loop complementarity. Precursor miRNAs may be produced based on known pre-miRNAs and pri-miRNAs or on mRNA sequences, where specificity is enhanced by increasing complementarity of at least some of the nucleotides in the loop. Also, mimetic precursor miRNAs may be produced where the seed sequence and the loop are directly joined or joined by other than a natural linker. Precursor miRNAs with improved target gene selectivity and/or modified activity may be selected from a library of precursor miRNAs molecules with randomized loop sequences through vitro and in vivo screening assay. Enhanced specificity in translational suppression is achieved with the subject precursor miRNAs and mimetics thereof, which may be provided by introduction into cells as a composition or introduced into cells as DNA for transcription of the precursor miRNAs. The subject precursor miRNAs may be used in arrays, where at least two precursor miRNAs, as may be modified, will have similar sequences, but stems and/or loops differing by at least one nucleotide.

Precursor miRNAs with unique loop nucleotides are screened for antisense oligonucleotides that complement to the loop region of the precursor miRNAs that can selectively silence miRNA genes which encode identical or nearly identical mature miRNAs.

The precursor miRNAs molecules may be redesigned to recognize novel sequences for repression. Either or both the stem or loop regions may be designed, where the loop regions are identified as enhancing selectivity for target mRNAs, whereby target mRNAs are determined based on complementarity to both the guiding sequence of the stem and at least 3 nt of the loop. Target mRNAs may be perfectly matched or be partly complementary to the guiding sequence of the stem and at least 3 nt of the loop. Synthetic or DNA encoded re-designed precursor miRNAs are prepared where the seed sequence and at least 2 nt of the loop are complementary to the target mRNA, desirably without bulges or interruptions between binding nucleotides.

### DESCRIPTION OF THE DRAWINGS

FIGS. 1A, 1B, 1C and 1D. The OP9-DL1 co-culture assay for measuring the effects of miRNA ectopic expression on T cell differentiation. (A) Schematics depicting the OP9-DL1 stromal co-culture assay for T cell differentiation. (B) Box-plots to summarize the effects of mir-181a-1 on the percentage of DP cells differentiated from DN progenitor cells. The results of a representative OP9-DL1 stromal co-culture assay (12 independent replicates for each construct) are shown. (C) Normalized box-plots. (D) Representative FACS plots showing the effects of mir-181a-1 on DP cell development (gated on infected GFP cells).

FIGS. 2A, 2B, and 2C. The pre-miR-181a-1 loop nucleotides control the distinct activities of the mir-181a-1 and mir-181c genes in DP cell development. (A) Nucleotide sequences of mature miR-181a (SEQ ID NO:60) and miR-181c (SEQ ID NO:61). (B) Schematics and nucleotide sequences depicting the wild-type mir-181a-1 and mir-181c genes (SEQ ID NOS:62 and 63) and corresponding precursors. Also shown are the chimeric miRNA genes, with the mature miRNAs (SEQ ID NOS:64-67), pre-miRNAs (SEQ ID NOS:63 and 62, respectively), and pre-miRNA loops (SEQ ID NOS:65 and 64, respectively) swapped between mir-181a-1 and mir-181c and termed “mature-chimeric”, “pre-chimeric”, and “loop-chimeric”, respectively. These mutant genes are designated as mir-181a (c stem), mir-181c (a stem), mir-181a (pre-181c), mir-181c (pre-181a), mir-181a (c-loop), and mir-181c (a-loop). (C) The effects of the chimeric mir-181a-1/c genes on DP cell development. Normalized data from 3-7 independent T cell assays (each with 12 independent replicates for a total of 36-84 replicates) are pooled and graphed in the distribution box plots. Mann-Whitney Rank Sum Tests were performed to determine whether the activities of the chimeric miRNA genes are statistically different from those of the negative control vector (*, p<0.0001) and/or mir-181a-1 positive control (*, p<0.0001). A representative OP9-DL1 stromal co-culture assay without normalization is also shown (FIG. 13).

FIGS. 3A and 3B. The activity of the mir-181a-1 gene in DP cell development is highly sensitive to nucleotide changes in the pre-miR-181a-1 loop region. (A) Schematics of the pre-miR-181a-1 loop mutants (SEQ ID NOS:68-73), with the wild-type sequence shown on top (SEQ ID NO:62). Two nucleotides at a time around the pre-miR-181a-1 loop were mutated (red nucleotides), while the mature miR-181a nucleotides remained constant (boxed nucleotides). (B) The effects of pre-miR-181a-1 loop mutants on DP cell development. Normalized data from at least six independent T cell assays (each with 12 independent replicates for a total of 72 replicates) are pooled and graphed in the distribution box plots to summarize the relative activities of mir-181a-1 and its loop mutants in DP cell development. Mann-Whitney Rank Sum Tests were performed to determine whether the activities of the loop mutants are statistically different from those of the negative control vector (*, p<0.0001) and/or the mir-181a-1 positive control vector (*, p<0.0001). A representative OP9-DL1 stromal co-culture assay without normalization (12 independent replicates for each construct) is also shown (FIG. 14).

FIGS. 4A, 4B and 4C. Mature miRNAs produced from the mir-181a-1/c mutants have the same 5′ ends, as indicated by primer extension analyses. Gel separations are shown for the different modified miR-181s using miR-181a as a probe in FIGS. 4A and 4C, and miR-181c as a probe in FIG. 4B

FIGS. 5A, 5B, 5C, 5D, 5E and 5F. The effects of mir-181a-1/c mutants on mature miRNA expression in BOSC23 and DP cells. (A, B) Gels providing the copy numbers of mature miR-181a (A) and miR-181c (B) expressed in BOSC23 cells transfected with the same amounts of various viral vectors expressing different mir-181a-1/c mutants, determined by quantitative Northern blot analyses (See also FIG. 15) (C, D) The copy numbers of mature miR-181a (C) and miR-181c (D) expressed in DP thymocytes transduced with viral vectors expressing various mir-181a-1/c mutants, determined by miRNA qPCR analyses. (E) A gel providing the copy numbers of mature miR-181a expressed in BOSC23 cells transfected with the same amounts of various viral vectors expressing unique mir-181a-1 loop mutants, determined by quantitative Northern blot analyses (See also FIG. 16). (F). The copy numbers of mature miR-181a expressed in DP thymocytes transduced with viral vectors expressing various mir-181a-1 loop mutants, determined by miRNA qPCR analyses. Statistical significance was determined by an unpaired two-tailed student's t test (compared to the control vector, *, p<0.05). Representative blots of four or more independent quantitative Northern blot analyses are shown (A, B, E).

FIG. 6. A “heat map” of the functionally important nucleotides in the pre-miR-181a-1 region according to mutagenesis analyses. (FIG. 1-3). Shading was used to illustrate the importance of the pre-miRNA nucleotides to the activity of the miR-181a-1 gene (SEQ ID NO:62), the more darkly shaded the greater the contribution to selectivity. Possible mechanisms by which pre-miRNA loop nucleotides control the activities of miRNA genes are also listed. The mRNA target stem and loop sequences are also shown as SEQ ID NOS:74 and 75, respectively.

FIGS. 7A, 7B and 7C. Effects of the mutations in the stem region on mir-181a-1 activity in promoting DP cell development. (A) Scanning mutations in the stem region of the mir-181a-1 gene (SEQ ID NO:74). The mutations are shown in the sequences listed below the mir-181a-1 gene and correspond to SEQ ID NOS:76-90, respectively. Two nucleotides (2-nt mutants, light shading) or a stretch of nucleotides (segment mutants, light shading) in the mature miRNA region are altered. Nucleotides are altered to disrupt their potential base pairing to target genes. Compensatory mutations are also generated on the miR* strand to maintain the secondary structure of the pre-miRNAs (See FIG. 8) (B) Expression and processing of wild-type mir-181a-1 and stem mutants. A gel is depicted where specific probes that perfectly match the mature miR-181a and each of its mutant forms were used in hybridization to determine the expression of mature miR-181a and its stem mutant forms. (C) The effects of mir-181a-1 and its stem mutants on DP cell development. Normalized data from 3-5 independent T cell assays (each with 12 independent replicates, total 36-60 replicates) are pooled and graphed in the distribution box plots to summarize the distribution of the relative activities of mir-181a-1, the 2-nt mutants, and the segment mutants in DP cell development. Mann-Whitney Rank Sum Tests were performed to determine whether the activities of individual 2-nt mutants were statistically different from those of the control vector (*, p<0.0001, or specifics p value indicated) and the mir-181a-J vector. A representative OP9-DL1 stromal co-culture assay without normalization (12 independent replicates for each constructs) is also shown (FIG. 12).

FIG. 8. Schematic and nucleotide sequences depict mature mir-181a-1 mutants. Compensatory mutations are introduced to maintain the integrity of the pre-miRNA secondary structure. (Indicated by shading and lower case.) These sequences correspond to SEQ ID NO:62 and SEQ ID NOS:91-105, respectively.

FIGS. 9A and 9B. Expression and processing of wild-type mir-181a-1 and the M1 stem mutant gene. (A) Nucleotide sequences of the wild-type miR-181a (SEQ ID NO:74) and the M1 mutant (SEQ ID NO:76). (B) Northern blot analyses of mature miRNA expression from the wild-type miR-181a and the M1 mutant. Total RNA was prepared from BOSC cells transfected with constructs expressing mir-181a-1, or the M1 mutant genes. Relative transfection efficiencies were determined by qPCR analyses of GFP mRNA levels produced from the transfected miRNA constructs, then used to normalize RNA loadings in Northern blot analyses. A shorter probe that perfectly matches to both mature miR-181a and the M1 mutant forms is used in hybridization to determine the expression of mature miR-181a and its mutant forms. Relative expression levels of the mature miRNAs determined by phosphoimager quantification is indicated.

FIGS. 10A and 10B. Members of the mir-181 gene family. (A) Alignment of the mature miR-181 miRNAs (SEQ ID NOS:106-109). (B) Schematics and nucleotide sequences depicting the pre-miRNAs of the mouse (SEQ ID NOS:110-115) and human (SEQ ID NOS:116-121) mir-181 gene family members. (Variations indicated by shading.)

FIG. 11. Developmental regulation of miR-181c expression in various purified thymocyte populations determined by miRNA qPCR.

FIGS. 12A and 12B. The effects of mutations in the mature miRNA region of the mir-181a-1 genes on DP cell development (See FIG. 7). (A) Box-plots summarize the percent of DP cells generated from DN progenitor cells infected with mir-181a-1, or mature miRNA mutant genes (gated on GFP positive). A representative OP9-DL1 stromal co-culture assay (12 independent replicates for each construct) is shown. The ends of the boxes define the 25^{th} and 75^{th} percentiles, a line indicates the median, and bars define the 5^{th} and 95^{th} percentiles. (B) Statistical summary. Mann-Whitney Rank Sum Tests were performed on this representative data set to determine whether the activity of mir-181a-1, mir-181c, or their chimeric mutants is statistically different from the control vector or the mir-181a-1 vector.

FIGS. 13A and 13B. The effects of the chimeric mir-181a-1 and mir-181c genes on DP cell development (See FIG. 2). (A) Box-plots summarize the percent of DP cells generated from DN progenitor cells infected with mir-181a-1, mir-181c, or their chimeric mutants (GFP positive). A representative OP9-DL1 stromal co-culture assay (12 independent replicates for each construct) is shown. The ends of the boxes define the 25^{th} and 75^{th} percentiles, a line indicates the median, and bars define the 5^{th} and 95^{th} percentiles. (B) Statistical summary. Mann-Whitney Rank Sum Tests were performed on this representative data set to determine whether the activity of mir-181a-1, mir-181c, or their chimeric mutants is statistically different from the control vector or the mir-181a-1 vector.

FIGS. 14A and 14B. The effects of the pre-miR-181a-1 loop mutants on DP cell development (See FIG. 3). (A) Box-plots summarize the percent of DP cells generated from DN progenitor cells infected with mir-181a-1, or pre-miR-181a-1 loop mutant genes (GFP positive). A representative OP9-DL1 stromal co-culture assay (12 independent replicates for each construct) is shown. The ends of the boxes define the 25^{th} and 75^{th} percentiles, a line indicates the median, and bars define the 5^{th} and 95^{th} percentiles. (B) Statistical summary. Mann-Whitney Rank Sum Tests are performed on this representative data set to determine whether the activity of mir-181a-1 and pre-miRNA loop mutant genes is statistically different from the empty vector (negative control) or the mir-181a-1 expressing vector (positive control).

FIGS. 15A, 15B and 15C. Mature and pre-miRNA expression levels from the chimeric mir-181a-1 and mir-181c genes (See FIGS. 5A and 5B). Total RNA was prepared from BOSC cells transfected with constructs expressing mir-181a-1, mir-181c, and the chimeric mir-181a-1 and mir-181c genes. Since all miRNA vectors contain an independent GFP reporter, percentage cells that are GFP positive were determined by FACS analyses and used to control for variations in transfection efficiency. Quantitative Northern blot analyses were carried out to determine the expression of mir-181a-1, mir-181c, and the chimeric mir-181a-1 and mir-181c genes. Specific probes that perfectly match to mature miR-181a or miR-181c were used in hybridization to determine the expression of mature and pre-miRNA forms. Band intensities were determined by phosphoimager quantification and normalized to the levels of wild-type controls accordingly. (A) Standard Curves for miR-181a and miR-181c. (B) The copies of mature miR-181a and miR-181c in BOSC 23 cells transfected with mir-181a-1/c mutants determined by quantitative Northern blot analyses. Average results of four independent experiments were plotted. (C) Relative levels of pre-miR-181a and pre-miR-181c in BOSC 23 cells transfected with mir-181a-1/c mutants determined by Northern blot analyses. Average results of four independent experiments were plotted.

FIGS. 16A, 16B and 16C. Mature and pre-miRNA expression levels from the pre-miR-181a-1 loop mutant genes (See FIG. 5E). Total RNA was prepared from BOSC cells transfected with constructs expressing the mir-181a-1 loop mutant genes. Since all miRNA vectors contain an independent GFP reporter, percentage cells that are GFP positive were determined by FACS analyses and used to control for variations in transfection efficiency. Quantitative Northern blot analyses were carried out to determine the expression of the pre-mir-181a-1 loop mutant genes. A probe that perfectly matches to the mature miR-181a was used in hybridization to determine the expression of mature and pre-miRNA forms. Band intensity was determined by phosphoimager quantification and normalized to the levels of wild-type controls accordingly. (A) Standard Curves for miR-181a. (B) The copies of mature miR-181a in BOSC 23 cells transfected with mir-181a-1 loop mutants determined by quantitative Northern blot analyses. Average results of four independent experiments were plotted. (C) Relative levels of pre-miR-181a in BOSC 23 cells transfected with mir-181a-1 loop mutants determined by Northern blot analyses. Average results of four independent experiments were plotted.

FIG. 17. Phylogenetic comparison of pre-miR-181a-1 and pre-miR-181c loop sequences (SEQ ID NOS:122-143). The full genus and species names and their abbreviations are as follows: Danio rerio, dre; Fugu rubripes, fru; Homo sapiens, hsa; Gallus gallus, gga; Gorilla gorilla, ggo; Lagothrix lagotricha, lla; Macaca mulatta, mml; Mus musculus, mmu; Macaca nemestrina, mne; Pan paniscus, ppa; Pongo pygmaeus, ppy; Pan troglodytes, ptr; Rattus norvegicus, rno; Sus scrofa, ssc; Tetraodon nigroviridis, tni.

### DESCRIPTION OF THE SPECIFIC EMBODIMENTS

Precursor miRNAs, primarily pre-miRNAs and pri-miRNAs, specificity is identified with both the stem and loop of the pre-miRNA portion of about 65 nt±10 nt, optionally including the flanking sequence, usually about 60 nt, having a stem that may lack perfect complementarity between the two strands and the target and a loop of at least about 8 nt. Alternatively, synthetic precursor miRNAs or smaller fragments thereof comprising at least the stem and loop may be prepared where the stem and relevant members of the loop have enhanced complementarity to the target mRNA or perfect complementarity to the target, where the seed sequence of the stem and the relevant nucleotides of the loop are separated by a sequence which need not have perfect complementarity to the target.

In referring to the parts of the pre-miRNA contained in the pri-miRNA, the pre-miRNA has a stem and loop, the 5′-strand of the stem being the guide sequence and comprising the seed sequence, with the flanking sequences extending from the ends of the stem. For the purposes of this invention, in referring to miRNA precursors, it is intended both pri-miRNA and pre-miRNA and mimetics thereof. However, for the most part, the invention deals with pre-miRNA as a precursor to miRNA and processing of a limited number, preferably 1, target mRNA, although in some instances there will be an interest in the modified pre-miRNA providing for processing of a plurality of mRNAs, usually not more than 5, more usually not more than 3. Also, for expression in cells, the precursors will include a gene encoding RNA from which the pri-RNA is derived, or encoding the pri-RNA or pre-RNA, or extended sequences thereof, where the extended sequences do not interfere or are processed in a cell to an active pre-miRNA.

With the subject discovery that it is the pre-miRNA that must be included for enhanced specificity, new opportunities are provided for using RNAi for modulating protein expression and phenotype of cells. By providing for sequences in the stem and loop that enhance or diminish affinity for one or more target mRNAs, the use of RNAi is made more precise in selecting targets and affecting cellular pathways. Affinity with one or more mRNAs can be modified by substitution of nucleotides, use of unnatural sequences as the backbone, replacing the natural sugar phosphate backbone, deletions and insertions, changing the number of loop members and changing the secondary structure of the loop. In this manner one can run the gamut of no or little activity toward a particular mRNA to high specificity for a particular mRNA, as compared to mRNAs with which a naturally occurring mature miRNA would act.

mRNAs are identified as targets of the pre-miRNA by hybridizing the pre-miRNA with at least a portion of mRNA, usually the 3′-UTR of the mRNA, although introns may be employed, or DNA encoding such sequence and identifying the sequence that hybridizes with both the guiding sequence of the stem and at least 2 nt of the loop, where such sequences and loop nucleotides need not be contiguous, nor need the complementary sequences in the mRNA be contiguous. Identification may be achieved with individual pre-miRNAs and pri-miRNAs employing protocols that permit detection of the hybridization of the pre-miRNA with one or more mRNAs and identifying the mRNA(s) with the closest complementarity to both the stem and loop, by using arrays of miRNAs, where the precursor miRNAs, particularly pre-miRNAs, may have the same or similar stems, but will differ in at least one nucleotide in the loop, by comparison of the sequences of mRNAs and the pre-miRNA or by using algorithms designed to identify related miRNA and mRNA(s), or by the direct measuring of the repression of a target gene in a functional assay and random library screen.

Libraries of random modifications of wild-type precursor miRNAs are prepared, where the libraries are used in assays to detect changes in phenotype of cells, as arrays to identify mRNAs that bind, or identification of genomic sequences having the same sequence as the library member. These libraries will generally be limited to modifications in the loop sequence, the stem sequence or both. In addition to the random modifications, changes are made in the sequence to maintain the complementarity or lack of complementarity in the wild-type sequence. For example, where the modification in the loop results in there being complementarity between pairs of nucleotides on opposite sides of the loop, which complementarity did not previously exist, then the wild-type sequence would be further changed to remove the complementarity. In the stem, one will usually maintain the same level of complementarity between the 5′-seed sequence and complementary 3′-sequence, although maintaining the same complementarity or varying the complementarity is permissible. The number of permutations is not great and is readily achieved as shown in the Experimental section. Where one has a phenotype associated with a family of isoforms, by screening cells from which the wild-type miRNA (including precursors) has been identified, the ability to modify the phenotype of the cells can be evaluated. In one application, one modifies the seed sequence to have identical complementarity with the mRNA and randomly modifies the loop, using single or double replacements.

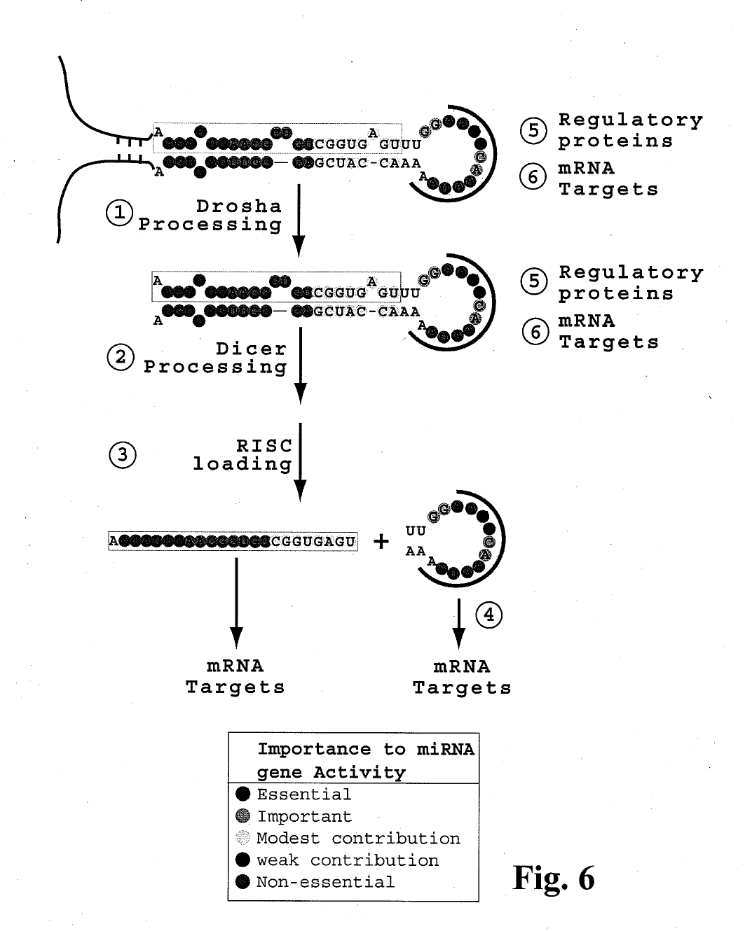

As partially shown in FIG. 6, starting with the 5′ terminus and counting from the first nucleotide that initiates the stem, at least the first 6 nucleotides are essential for binding to the target. The next 8 nucleotides may have varying degrees of complementarity to the target, from 0 to 8 being complementary, where there may be 0 to 3 bulges of 1 to 2 nucleotides in either sequence (miRNA and target sequences). (By bulge is intended that complementarity is enhanced by removing the nucleotides in the bulge.) The next 8 to 10, particularly 8, nucleotides include at least some degree of complementarity, generally there being at least 2, usually at least 4, complementary nucleotides with the target and there may be up to 10 complementary nucleotides including from 0 to 3 bulges in either sequence to provide the complementarity. The first 2 members of the loop are important in matching the target and the 7^{th} and 8^{th} nucleotides can affect the specificity, with lesser effect from the 5 and 6^{th} nucleotides. The other nucleotides in the loop are less significant in determining specificity.

For synthesized precursor miRNA, where complementarity to the target mRNA is enhanced, the pre-miRNA will have the following formula:

(SEQ ID NO:1)

5′-Xₙ₁-Sₙ₂-Yₙ₃-Zₙ₄-Lₙ₅-Z′ₙ₆-Y′ₙ₇-S′ₙ₈-X′ₙ₉.

The subscript n intends the number of nucleotides in the particular portion of the pre-miRNA. X and X′ with the exception of the nucleotide joined to S or S′ respectively will be removed during processing of the pre-miRNA, where n¹ and n⁹ may be the same or different and will generally be from about 0 to the number of nucleotides present in the pri-miRNA, which may be as many as a thousand nucleotides or more. S is the seed sequence and S′ is the complementary sequence where n² and n⁸ may be same or different, preferably the same, and will generally be in the range of about 6 to 10, preferably 6 to 8, and more preferably 6, where there may or may not be perfect complementarity between S and S′, there generally not being more than 2 mismatches, usually not more than 1 mismatch. Y and Y′ are spacers and their sequence is less relevant, except that they should be chosen to avoid secondary structure with the other portions of the pre-miRNA. n³ and n⁷ will generally be from about 6 to 10, more usually about 7 to 9, preferably about 8. Z and Z′ are sequences where at least 2, preferably at least 3, and generally not more than all (10), usually not more than 8, are complementary to the mRNA target, including from 0 to 3 bulges in either sequence to provide the complementarity and not more than 3, usually not more than 2, and preferably not more than 1, mismatch. L is the loop sequence where the nucleotides are substantially mismatched to avoid secondary structure in the loop. n⁵ will be about 10 to 15, more usually 11 to 14, preferably 13. At least one of the first two nucleotides, preferably both will bind to the mRNA, while it is desirable that the 7^{th} and 8^{th} nucleotides also bind to the mRNA downstream from the seed sequence, as well as the 5^{th} and 6^{th} nucleotides of lesser relevance, where the intervening sequence between the seed sequence and the loop may have a plurality of bulges and mismatches, so that the complementary loop nucleotides need not be separated from the seed sequence by the same number of nucleotides that the complementary sequences of the target are separated.

The subject invention allows for improved prediction of the modulation of mRNA expression with miRNA. By including complementarity between the stem and a portion of the loop with the candidate mRNA, one will be able to better predict whether the miRNA will affect the expression of the mRNA. One may include the sequence of the loop as a modification of the algorithm or use the presently available or future algorithms and then compare the adjacent nucleotides of the mRNA with the nucleotides of the loop sequence. See, for example, U.S. Patent application nos. 2007/0100563 and 2007/0099196, for methods of designing miRNA molecules and predicting mRNA targets, the disclosure of which is specifically incorporated herein by reference. Greater homology between indicated portions of the loop sequence and the nucleotides in the mRNA proximal to the sequence complementary to the seed sequence will indicate the greater likelihood of regulation of the mRNA by the pre-miRNA.

One can define a particular RNA sequence based on algorithm predictions, where both the stem and the loop sequences are included in the analysis. By introducing such subject miRNA precursors into cells as the precursor or the gene, one can determine the effect on the phenotype of the cell. A change in phenotype indicates that the subject miRNA precursors have an effect in the degradation or storage of the target mRNA(s). In addition, one may search the sequence database for mRNA sequences, particularly 3′-UTR sequences, that have substantial complementarity to the seed sequence and at least 2, preferably at least 3, nt of the loop sequence to identify mRNAs that are likely to be regulated by the subject miRNA precursors. Most of the loop sequence need not be complementary, desirably up to 6 nt, where bulges of 1 to 3 nt and mismatches are permitted. Where the function of the mRNAs is known, the regulatory effect of the miRNA will then also be known. Thus, one determines the sequence of an mRNA complementary to at least the seed sequence of a stem sequence of a mature miRNA, where the seed sequence will generally be of from about 6 to 10 nucleotides of the 5′ strand. One would also include in the analysis a sequence of at least 2, preferably 8, nucleotides of the loop sequence for complementation of the first two and last two of the 8 nt to a sequence of at least a comparable number of nucleotides of said mRNA sequence that is proximal to said mRNA seed complementarity sequence. Desirably, the complementary nucleotides in the loop and the mRNA will be equally spaced apart, so that there will be no bulges, although there may be mismatches.

While not being bound to any theory, it would appear that the loop of the precursor miRNA is involved in binding to the mRNA and may initiate the binding of the precursor miRNA to the target mRNA, followed by the mRNA invading the stem and displacing the 5′-strand while binding to the 3′-strand.

The subject invention finds employment with any source of pre-miRNA, both prokaryotes and eukaryotes, animals, including vertebrates, insects, fish, etc., more particularly mammals, e.g. primates, rodents, domestic animals, etc., unicellular eukaryotes, plants, and the like. Cells, tissues and whole mammals, including human, may be modified by introducing a gene encoding a mutated pre-miRNA according to the subject invention in such cells, whereby the transcriptional regulatory region is selected to be functional in the host cells.

The precursor mi-RNAs of this invention may be used in the modulation of numerous cellular pathways. Pathways of interest include cellular division, immune response, stress response, injury response, hormone secretion, synthesis of non-proteins, glucose response, organ development, differentiation, etc.

The phenotype of a cell can be modified with greater specificity by employing a particular precursor miRNA that acts on a single target in a pathway of interest, acts on a plurality of targets while excluding other targets, or acts with greater efficiency on one or more targets, particularly where the targets may be in single or related pathways. Using loop sequences that bind to selected target(s), particular a single target, the modulation of the cellular pathway can be more precisely controlled. The pre-miRNA can be matched with a particular mRNA or small number of mRNAs, usually not more than 5, more usually, not more than 3, mRNAs.

Of particular interest is the use of modified precursor miRNAs. The modifications may be as to sequence, backbone, chemical conjugation, use of unnatural bases, deletions, insertions, etc. The purpose(s) of the modifications may be to enhance affinity, reduce degradation by nucleases, prevent or enhance cross-reactivity, permit ready identification of hybridization, etc. Where the precursor miRNA is naturally cellularly expressed, then the modifications will usually be limited to sequence modifications, rather than modifications involving substitution of bases with entities that bind to the same complementary base.

As indicated above, the sequence modifications may take many forms. Where the pre-miRNA is produced by cellular expression, then differences will be as to the sequence, which will involve deletions, insertions and substitutions. Modifications can be selected to allow for greater or lesser complementarity between the two sequences of the stem. With 6 to 8 nucleotides of the guide sequence complementary to the target mRNA, the binding of a second portion of the same strand to the mRNA is not required for repression. However, for fewer nucleotides than 6 complementary to the mRNA, then the second portion will usually be involved. Once the mRNA sequence that binds the miRNA guide sequence is known, one can enhance affinity by providing for greater complementarity between the guide sequence and the mRNA sequence, up to perfect complementarity. Where the pre-miRNA is synthesized, one may use modified nucleotides that provide for higher affinity between the guide sequence and the mRNA sequence. Various unnatural bases may be used, such as phosphorothioates, phosphorodithioates, polyamido(peptide) or polyamino backbones, modified sugars, e.g. LNA, modified bases, etc.

The mimetic molecules may be varied in different manners. The seed sequence and the complementary sequence in the loop will usually have a linking group of up to 20 nucleotide units, more usually not more than about 18 nucleotide units, and at least about 16 nucleotide units. In some instances other than nucleotides or nucleotide mimetics may be employed as the linker, where there will generally be from about 54 to 120 atoms in the chain, usually from about 60 to about 108 atoms in the chain, where a ribose phosphate is counted as 6 atoms, an amide as 3 atoms, etc. The particular spacer will be selected to provide the optimum activity of the pre-miRNA in repressing translation. The linking group may be a naturally occurring linking group from a naturally occurring pre-miRNA binding to the target mRNA, a truncated naturally occurring linking group, truncated by from 1 to 6 nucleotides, may be a poly-U or -A or combination thereof, random, alternating or block, abasic nucleotides, or portions of one with another. The linker may be varied widely providing for minimal interference with the binding of the pre-miRNA with the target mRNA, minimizing cross-reactivity with non-target mRNA, avoiding false positives and negatives, and providing for optimum binding of the seed sequence and the loop sequence with the target mRNA.

The loop sequence beginning with unpaired nucleotides at the end of the stem will generally be from about 3 to 18 nucleotides, more usually from about 4 to 15 nucleotides, frequently at least 8 nucleotides and up to the upper limits indicated above, where the loop sequence will include at least the sequence of nucleotides binding to the target mRNA. The loop sequence that binds will have at least 2 contiguous nucleotides that are complementary to the target mRNA, and may have at least 4 or more, usually not more than about 10, more usually not more than about 8, and conveniently not more than about 6, that are complementary to the target mRNA, where beyond 2 nt, the nucleotides of the loop and the target that are complementary need not be contiguous. The binding loop sequence may have from 0 to 12 mismatches with the target mRNA sequence. There may be deletions or bulges of 1 to 2 nt in the target mRNA to provide for complementarity. The nucleotides of the loop of primary interest counting from the 5′ end are 1, 2, 7, and 8, with 5, and 6 being of secondary interest.

The pre-miRNAs may be prepared by transcription of mutated genes, using transcription constructs having an appropriate transcription regulatory region as described in the references cited above. The genes may be for pri-miRNA or pre-miRNA or other precursor to pre-miRNA. The miRNA precursor gene may be a separate gene or intronic. The gene may be introduced into a cellular host as bare DNA, plasmid, viral vector or the like. For preparation of the miRNA precursor, either prokaryotic or eukaryotic hosts can be employed. Alternatively, the pre-miRNA can be synthesized using commercially available synthesizers in known ways. Since in some cases, the subject miRNAs will have fewer than the naturally occurring pri-miRNAs, the synthesis will be easier and provide for a purer product. Of course, one can isolate a wild-type gene and by appropriate manipulation modify the nucleotides to obtain the desired sequence.

The subject pre-miRNAs may be divided into linear strands or stem and loop strands. The linear strands would comprise at least the seed sequence and at least the binding portion of the loop and a linker between the seed sequence and the portion of the loop or would have a stem and loop, where the 3′ strand of the stem would be at least partially complementary to the 5′ strand, usually having fewer than 8 mismatches total between the two strands, e.g. bulges, deletions and insertions, where the 3′ strand may also be at least partially complementary to a sequence in the mRNA target sequence.

Instead of having a single nucleic acid complementary to putative mRNAs, one may have two different complementary nucleic acids: one comprising at least the complementary seed sequence and preferably at least about 80% of the guide stem; and a second complementary at least to the binding portion of the loop sequence up to the entire loop sequence. One could add both of these sequences together for hybridization to pri- or pre-miRNA or preferably separately. By using the two components of the pre-miRNA one can screen for the miRNA precursors as to hybridization for the seed sequence followed by hybridization for one or more loop sequences or vice-versa, screen for the hybridizing loop sequences, followed by screening for the seed sequence. This offers many opportunities for unraveling the presence of similar miRNA precursors, where one is interested in the role of the loop in regulating expression and/or the role of the stem in regulating expression. The loop sequence used for capturing the miRNA precursor will be at least about 6 nt, more usually at least about 8 nt and up to the entire loop. In some instances it may also include up to 3, usually not more than about 2 nt of the nucleotides in one or each of the strands adjacent to the loop.

Depending upon the application for the subject pre-miRNAs, the subject pre-miRNAs may take many forms. For example, there are the uses of the pre-miRNAs in arrays. The arrays may be used for research purposes, where one is attempting to identify miRNAs from a host. In this case one might take a known stem and vary the loop one or two nucleotides at a time, where the entire possible population of nucleotides would be presented. In this manner one would identify numerous mRNAs that would bind to the seed sequence, which mRNAs might have a variety of different sequences, varying as to the sequence that binds to the loop. Alternatively, one may wish to assay for a transcriptome of the pre-miRNAs, so that the sequences employed in the array would be complementary to known pre-miRNAs or pre-miRNAs of known stems, but possibly unknown loops. By providing for hybridization between the bound sequences and pre-miRNAs binding to such sequences in the array, one could determine both qualitatively and quantitatively, the pre-miRNAs that are present in cells, tissue or the like. By having sequences where the sequences have a higher binding affinity to target mRNAs than the natural mature miRNAs by virtue of sequence modifications, one obtains a more accurate transcriptome as a result of lower levels of cross-reactivity.

As described in the references cited herein and many other articles in the literature, there are numerous methods for detecting binding between two nucleic acid strands. By having a stem, a fluorescer and a quencher can be bound at proximal sites on opposite strands, so that binding opens the stem and allows for fluorescence. One can use DNA fixed strands that are labeled at the unbound terminus, where by treating DNA-RNA hybrids with a DNA-RNA nuclease, the label is released into the supernatant and can be measured. One can bind the sample mRNAs or pre-miRNAs to a surface and interrogate the bound RNA with labeled subject pre-miRNAs or mimetic sequences and measure the label that is bound. The particular method of determining binding is not crucial to this invention and any convenient method providing for the appropriate specificity and sensitivity can be employed.

The terms “specific binding”, “specifically bind”, or other like terms, refer to the ability of a capture agent to preferentially bind to a particular target that is present in a mixture of different molecules, normally including one or more target molecules. Desirably, there will be discrimination between target molecules and non-target molecules of about 10 to 100-fold or more. Frequently, the binding constant between the capture pre-miRNA and target RNA or capture pre-miRNA and target miRNA is greater than 10⁶M⁻¹, and usually not more than about 10¹⁰M⁻¹. “Specific binding conditions” are conditions sufficient to allow a capture agent to preferentially bind to a particular analyte, e.g. stringent assay conditions. Stringent assay conditions are the summation or combination (totality) of both hybridization and wash conditions.

A “stringent hybridization” and “stringent hybridization wash conditions” in the context of nucleic acid hybridization (e.g., as in Southern or Northern hybridizations, or hybridization of molecules in solution, or in array assays) are sequence dependent, and are different under different experimental conditions. Hybridization conditions are well known to those of skill in the art. Illustrative stringent hybridization conditions, in solution or with the nucleic acid bound to a surface, that can be used to identify nucleic acids within the scope of the invention may include, e.g., hybridization in a buffer comprising 50% formamide, 5×SSC, and 1% SDS at 42° C., or hybridization in a buffer comprising 5×SSC and 1% SDS at 65° C., both with a wash of 0.1×SSC and 0.1% SDS at 37° C. a buffer of 40% formamide, 1 M NaCl, and 1% SDS at 37° C., and a wash in 1×SSC at 45° C. 0.5 M NaHPO₄, 7% sodium dodecyl sulfate (SDS), 1 mM EDTA at 65° C., and washing in 0.1×SSC/0.1% SDS at 68° C., 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5.

The particular protocols will vary depending upon the source of the RNA, the agents involved in the assay, the specificity and required sensitivity, the employed label, the available equipment and the like. Usually, one of the members of the binding pair will be associated with a surface, either covalently or non-covalently bound. For covalent binding the subject pre-miRNAs or mimetics thereof may be prepared bound to a surface by a convenient linking group, particularly a particle surface, and the particles used in the assay. Alternatively, the subject pre-miRNAs may be functionalized, usually at a terminus and with a short linker, generally not more than 30 atoms in the chain, so as to be able to react with a functionalized solid surface that may be planar or spherical. Arrays can be prepared on microparticles, planar plastic and metal surfaces, etc. Conveniently, ink jet printing can be employed to ensure the spatial integrity of the different nucleic acid entities. Various functional groups can be used that are compatible with the nucleic acids, such as thiols, disulfides, activated olefins, amines, oxo- and non-oxo-carbonyl, etc.

Depending on the source of the sample, the sample may be pretreated before being assayed. From a cellular source, the RNA is isolated, purified and separated by size, e.g. gel electrophoresis. (Yi, et al. 2002 RNA 8, 180-7; Yi, et al. 2003 Genes Dev 17, 3011-6). The band having the desired molecular weight is then extracted from the gel and may be used for further studies.

The subject invention can be used in substantially all of the ways that have been taught for precursor-miRNA and miRNA with the added advantage that there will be fewer target mRNAs that are affected by employing a pre-miRNA that has a higher affinity for a narrower range of mRNAs, particularly as a result of a defined loop sequence that defines a particular target mRNA. Thus, the subject precursor-miRNAs, mimetics or fragments thereof, can be used in investigations of cellular pathways, the effect of modulation on cellular phenotype, modulation of cellular properties, both healthy and diseased cells, screening of pre-miRNA profiles, with greater specificity as to targets and their relationship to cellular states, e.g. diseased, propensity for particular diseases or mutations, evaluate particular proteins and pathways as to the state of cells, e.g. level of development, response to external stimuli, such as drugs, effect of protein expression modulation on the response of cells to drugs, and the like.

Toward this purpose cells may be prepared that are transiently or permanently modified with a genetic construct having a gene directly (encoding only the pre-miRNA) or indirectly (encoding a precursor to the pre-miRNA) coding for pre-miRNA and having transcriptional regulatory sequences functional in the host cell, so as provide either inducibly or constitutively, the encoded pre-miRNA. These cells may be used in culture to study the effect of external agents in the presence and absence of the pre-miRNA on the pathway in which the protein modulated by the pre-miRNA is active.

The subject invention also allows for the determination of the effect of changes in the loop sequence, such as isoforms of miRNA families. Methods such as exchanging all or part of loop sequences between the isoforms, substituting one or two nucleotides in a nucleotide walk using the different combinations of nucleotides, while avoiding pairing that results in new secondary structure within the loop, provides information about what the regulatory role of the subject miRNA precursors is and can provide new regulatory pre-miRNAs.

One can also identify the role of a loop sequence of a pre-miRNA among a plurality of isoforms differing at least in their loop sequences. One can substitute a portion of one loop from one isoform with a comparable portion of a loop from a different isoform. The first isoform and the modified isoform may then be introduced in separate cells of the same type and the phenotype of each of the cells determined. Where the effect on the phenotype of the pre-miRNAs is the same, one can conclude that the loop does not affect the phenotype, while if the phenotypes are different, then the loop isoform affects the phenotype for the two isoforms.

The subject precursor-miRNAs, that is, modified precursor-miRNAs, can be used in investigating more accurately the development of an embryo, stem cell or differentiated cell. By introducing a gene that expresses the pri- or pre-miRNA constitutively or inducibly, one can observe the effect on the cells and their development when the expression of a particular protein is modulated. One can introduce the gene into an intron, so as to have concomitant expression of the protein and the pre-miRNA. One can coordinate expression of the pre-miRNA with the expression of one or more proteins, where the same transcription factors for the expression of the pre-miRNA and protein(s) are the same, that is, the transcriptional regulatory regions respond to the same transcription factors.

One can define targets for treating diseased states by introducing the pre-miRNA directed to a specific protein to see the effect of the modulation of the expression of the protein has on the diseased state and the cells response to drugs.

Instead of having binding to mRNA, antisense pre-miRNA and pri-miRNAs may be used to investigate the role of the pri- and pre-miRNA in a cell or the role of the protein(s) that are suppressed by the pre-miRNA. The antisense sequence will bind to at least the seed sequence and a portion of the loop, preferably the 3′-stem and at least half of the loop proximal to the 5′-terminus of the stem. One compares cells that express the pre-miRNA by itself or together with the antisense with differences being associated with the presence and absence of pre-miRNA activity.

The following examples are offered by way of illustration and not by way of limitation.

### EXPERIMENTAL

### Nucleotide Sequences of miRNA Genes Used in this Study

(1) The Nucleotide Sequences of Wild-Type mir-181a-1 Gene

(SEQ ID NO:2)

CTCGAGtgtgacaggtttggttaaaggattgggctttcctctgcctccct
cctgctccagactcccacagatactgtttaaatcagcacatctctgcctc
acaggttgcttcagtgAACATTCAACGCTGTCGGTGAGTttggaattcaa
ataaaaACCATCGACCGTTGATTGTAccctatagctaaccatcatctact
ccatggccctctgcgtttgctgaagacagaaccgcaaagcaggacccgac
aggattcttttttaattaagaattcctagGaattcTTGCCAAACCTACAG
GTGGGGTCTTTCATTCCCCCCTTTTTCTGGAGACTAAATAAAATCTTTTA
TTTTATCGATaagcttGGCTGCAGGTCGACgcggccgc

(2) Pre-miRNA Nucleotide Sequences of the mir-181a-1 Mature and Loop Mutants
I. Wild-Type pre-miR-181a-1 Precursor

(SEQ ID NO:3)

AACATTCAACGCTGTCGGTGagtttggaattcaaataaaaACCATCGACC
GTTGATTGTA

II. 2-nt Mature miR-181a-1 Mutants

### M1:

(SEQ ID NO:4)

AtaATTCAACGCTGTCGGTGAGTttggaattcaaataaaaACCATCGACC
GTTGATTtaA

### M2:

(SEQ ID NO:5)

AACtaTCAACGCTGTCGGTGAGTttggaattcaaataaaaACCATCGACC
GTTGAaaGTA

### M3:

(SEQ ID NO:6)

AACATaaAACGCTGTCGGTGAGTttggaattcaaataaaaACCATCGACC
GTTttTTGTA

### M4:

(SEQ ID NO:7)

AACATTCttCGCTGTCGGTGAGTttggaattcaaataaaaACCATCGACC
GaaGATTGTA

### M5:

(SEQ ID NO:8)

AACATTCAAacCTGTCGGTGAGTttggaattcaaataaaaACCATCGACg
tTTGATTGTA

### M6:

(SEQ ID NO:9)

AACATTCAACGaaGTCGGTGAGTttggaattcaaataaaaACCATCGACC
GTTGATTGTA

### M7:

(SEQ ID NO:10)

AACATTCAACGCTcaCGGTGAGTttggaattcaaataaaaACCATCGtgC
GTTGATTGTA

### M8:

(SEQ ID NO:11)

AACATTCAACGCTGTacGTGAGTttggaattcaaataaaaACCATgtACC
GTTGATTGTA

### M9:

(SEQ ID NO:12)

AACATTCAACGCTGTCGcaGAGTttggaattcaaataaaaACCtgCGACC
GTTGATTGTA

### M10:

(SEQ ID NO:13)

AACATTCAACGCTGTCGGTctGTttggaattcaaataaaaACgATCGACC
GTTGATTGTA

### M11:

(SEQ ID NO:14)

AACATTCAACGCTGTCGGTGAcattggaattcaaataaaatgCATCGACC
GTTGATTGTA

III. Segment Mature miR-181a-1 Mutants

SM1:

(SEQ ID NO:15)

AtataaaAACGCTGTCGGTGAGTttggaattcaaataaaaACCATCGACC
GTTtttataA

SM2:

(SEQ ID NO:16)

AACATTCttacaacaCGGTGAGTttggaattcaaataaaaACCATCGtgg
taaGATTGTA

SM3:

(SEQ ID NO:17)

AACATTCAACGCTGTaccactcattggaattcaaataaaatggtggtACC
GTTGATTGTA

SM4:

(SEQ ID NO:18)

AACATTCttacaacaaccactcattggaattcaaataaaatggtggttgg
taaGATTGTA

IV. miR-181a-1 Loop Mutant

### LP1:

(SEQ ID NO:19)

AACATTCAACGCTGTCGGTGAGTttCCaattcaaataaaaACCATCGACC
GTTGATTGTA

### LP2:

(SEQ ID NO:20)

AACATTCAACGCTGTCGGTGAGTttggTTttcaaataaaaACCATCGACC
GTTGATTGTA

### LP3:

(SEQ ID NO:21)

AACATTCAACGCTGTCGGTGAGTttggaaAAcaaataaaaACCATCGACC
GTTGATTGTA

### LP4:

(SEQ ID NO:22)

AACATTCAACGCTGTCGGTGAGTttggaattGTaataaaaACCATCGACC
GTTGATTGTA

### LP5:

(SEQ ID NO:23)

AACATTCAACGCTGTCGGTGAGTttggaattcaCGtaaaaACCATCGACC
GTTGATTGTA

### LP6:

(SEQ ID NO:24)

AACATTCAACGCTGTCGGTGAGTttggaattcaaaATaaaACCATCGACC
GTTGATTGTA

(3) The Nucleotide Sequences of the Wild-Type mir-181c Gene

(SEQ ID NO:25)

GATCCTCGAGgtgagtcaaaggggaccctggtttctctctcgtcccacat
gctctctgccttgctggcctactctcccaactccagttatccaagaactt
gccaagggtttgggggAACATTCAACCTGTCGGTGAGTttgggcagctca
gacaaaCCATCGACCGTTGAGTGGAccccgaggcctggaactgccacccg
tctaccccatccccaccctgtagacccgggagagccccaggcagcatccc
ctgcctcaggccacagcaaaggtcacaattGAATTCGATC

An In Vitro Assay for Measuring the Effects of mir-181 genes on DP Cell Development

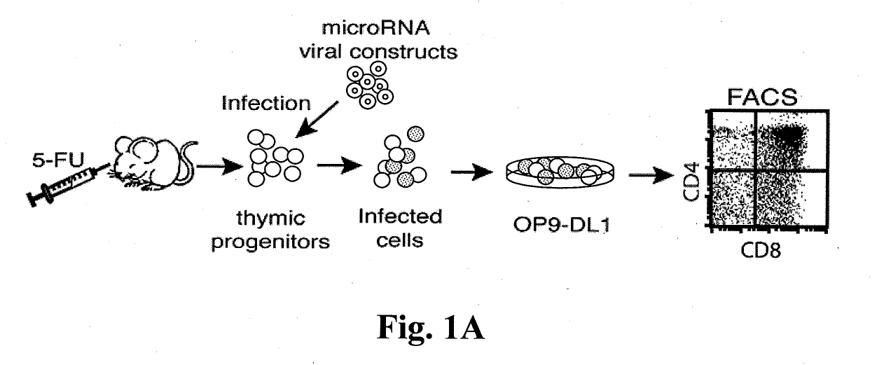

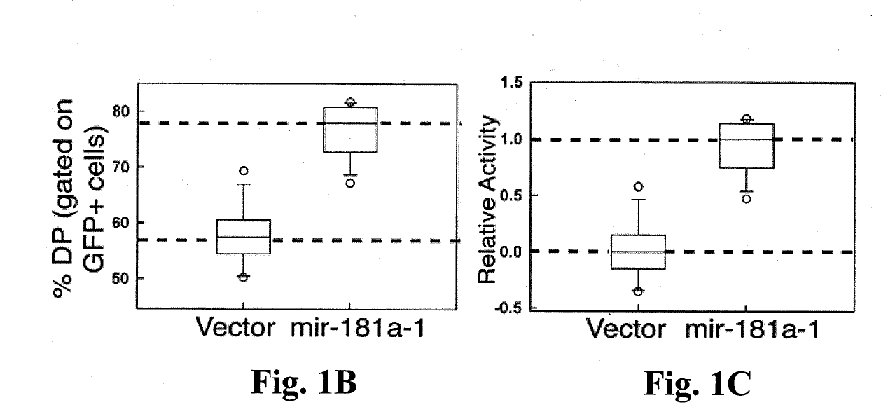

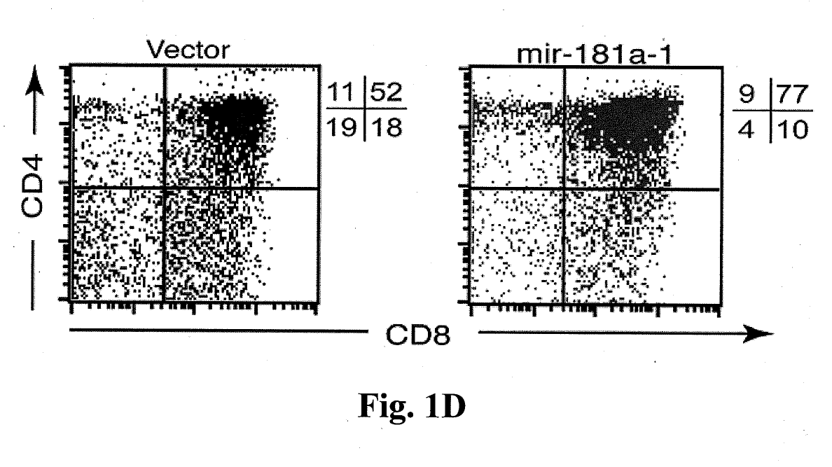

We used T cell development as a functional readout to determine the nucleotides and structural domains that are required for the function of mir-181 genes. mir-181a-1 plays important roles in T and B lymphocyte development (10-12), and can function as a rheostat to modulate the strength and threshold of T cell receptor (TCR) signaling (11). Moreover, mature miR-181a is developmentally regulated during early T cell differentiation, in the transition from CD4 and CD8 double-negative (DN) to CD4 and CD8 double-positive (DP) cells, in the thymus (11-13). Using the OP9-DL1 co-culture assay, which can recapitulate the differentiation of DN progenitors into DP cells in vitro (14), we showed that ectopic expression of mir-181a-1 in DN thymic progenitor cells lead to a significant increase in the percentage of DP cells, from a median level of ˜57% in the control group to a median level of 77% in the mir-181a-1 expressing group (FIG. 1). We have found that mir-181a-1 potentiates DN to DP cell development by targeting negative regulators in the Notch and pre-TCR signaling pathways. This assay allowed us to quantitatively measure the contribution of nucleotide sequences and structural domains to miRNA gene function via mutagenesis analyses.

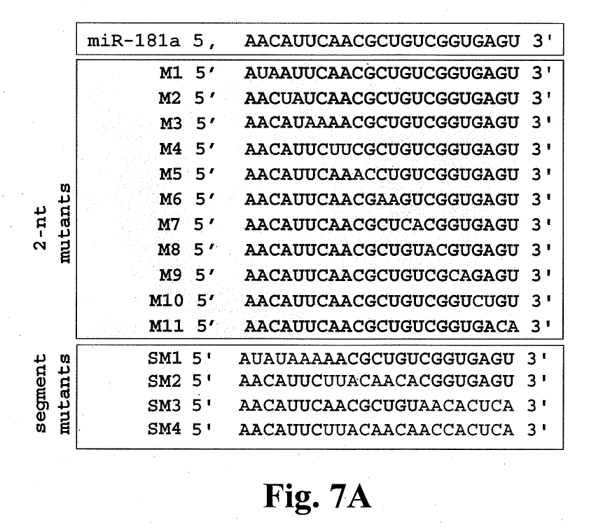

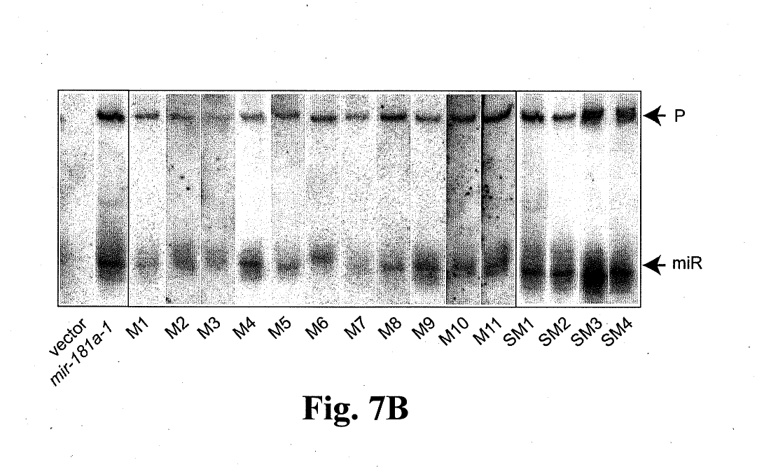

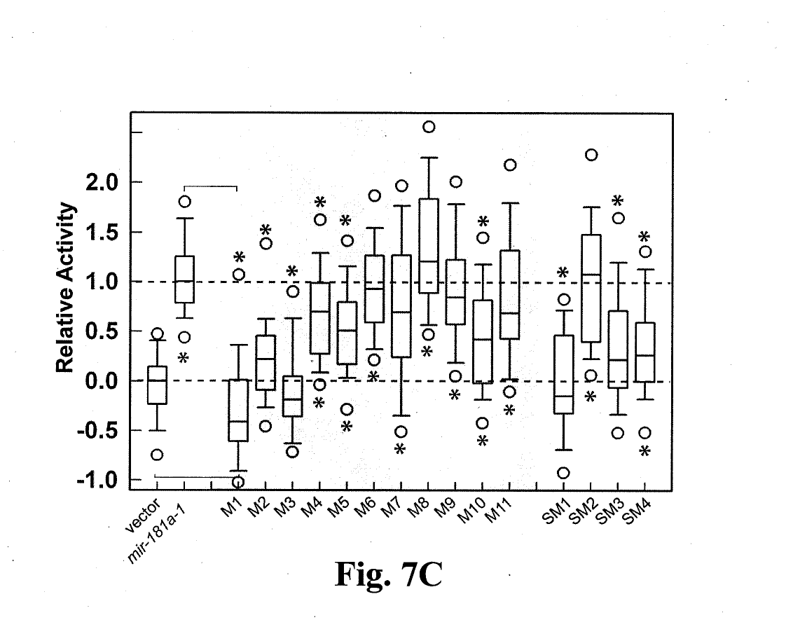

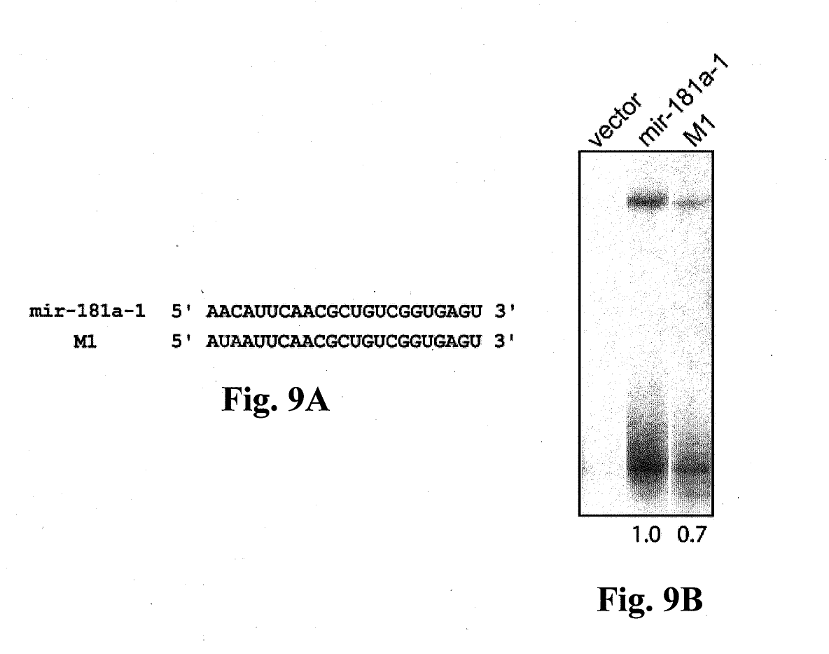

We generated “stem mutants” by systematically mutate the nucleotides in the mature miRNA region and then tested the activities of mir-181a-1 “stem mutants” in the OP9-DL1 co-culture assay. (FIGS. 7-10) We found that nucleotides in the 5′ seed region are critical for mir-181a-1 's activity in promoting DP cell development, whereas the nucleotides in the 3′ end of the mature miR-181a region make only minor contributions and the nucleotides in the center are not at all important to mir-181a-1 activity (FIGS. 7-10). These findings confirmed the importance of the seed nucleotides, shown previously by computational and biochemical analyses (6-8), thus validating the use of this assay to dissect the structural and functional relationships of mir-181 genes by mutagenesis.

mir-181a-1, but not mir-181c, can Promote DP Cell Development

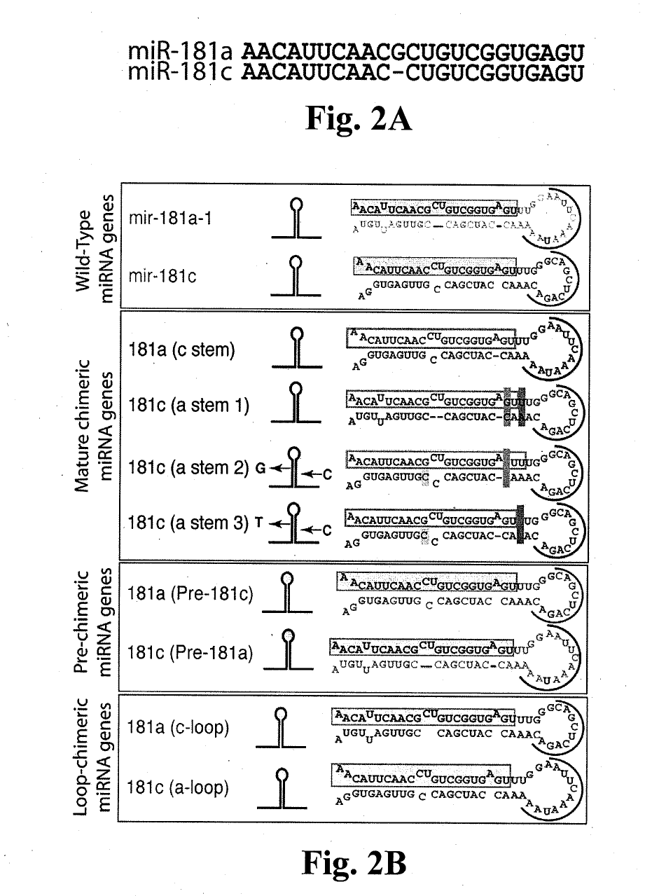

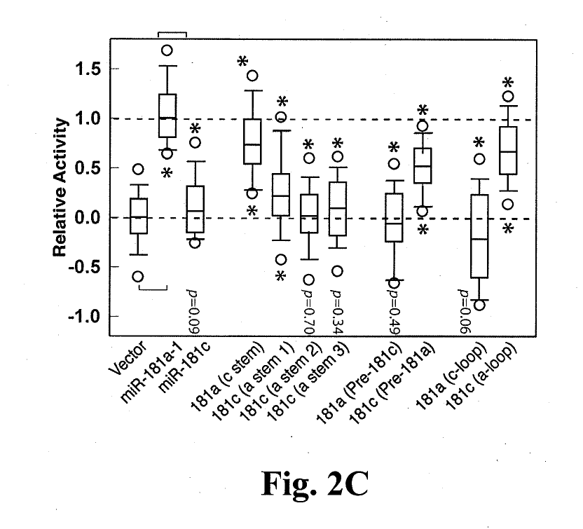

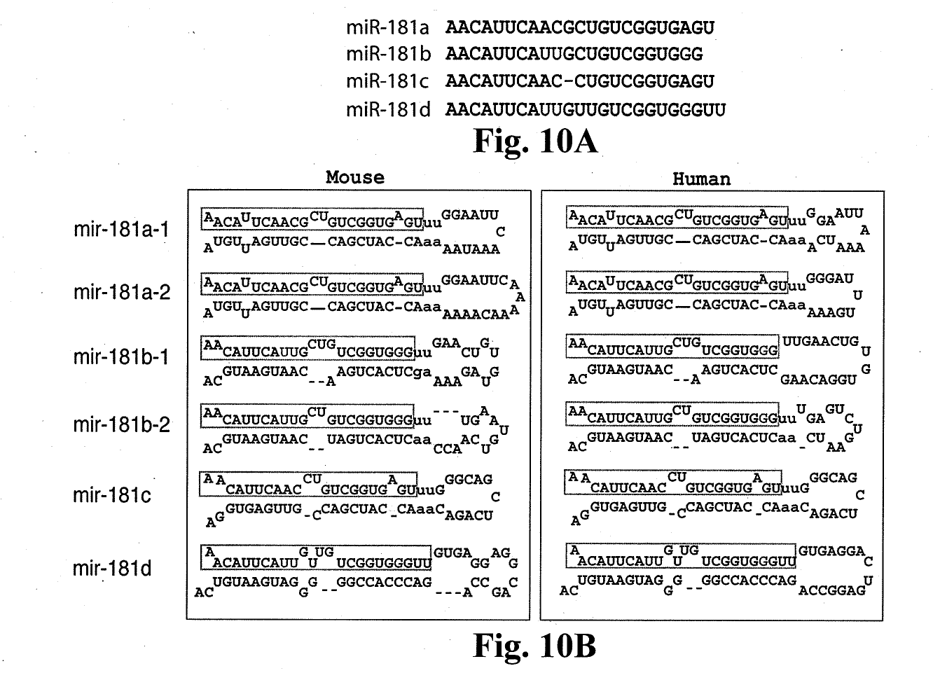

The members of mir-181 family of genes produce four mature miRNAs, miR-181a, miR-181b, miR-181c, and miR-181d, from three putative polycistronic transcripts, mir-181a-1/b-1, mir-181a-2/b-2, and mir-181c/d, respectively (FIG. 10). The mature miRNAs of the miR-181 family, all with identical 5′ seed nucleotides, differ from one another by no more than 3-nt in either the center or the 3′ end of the mature miRNAs. Particularly, mature miR-181a differs from miR-181c by only one nucleotide in the center of the mature miRNA (FIGS. 2A, 2B). Thus, according to the results of “stem mutant” analyses (FIGS. 7-10), it would be expected that mir-181a-1 and mir-181c should have similar activities in this co-culture assay.

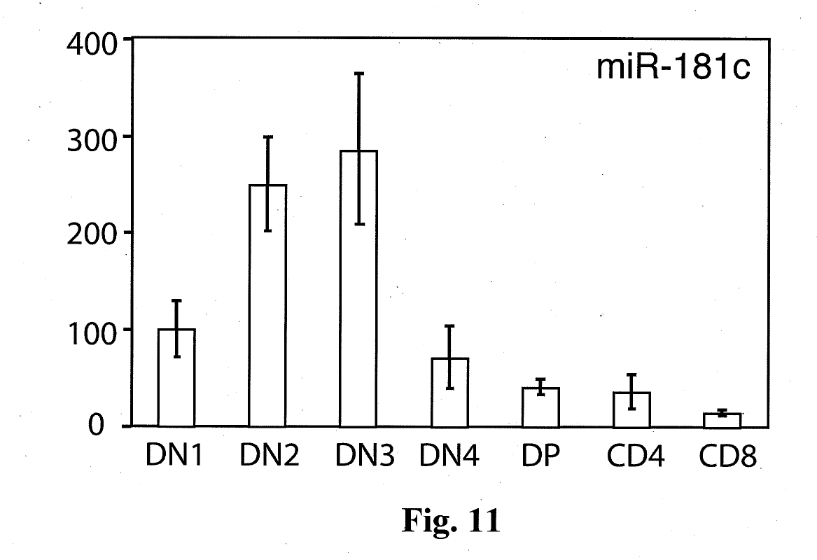

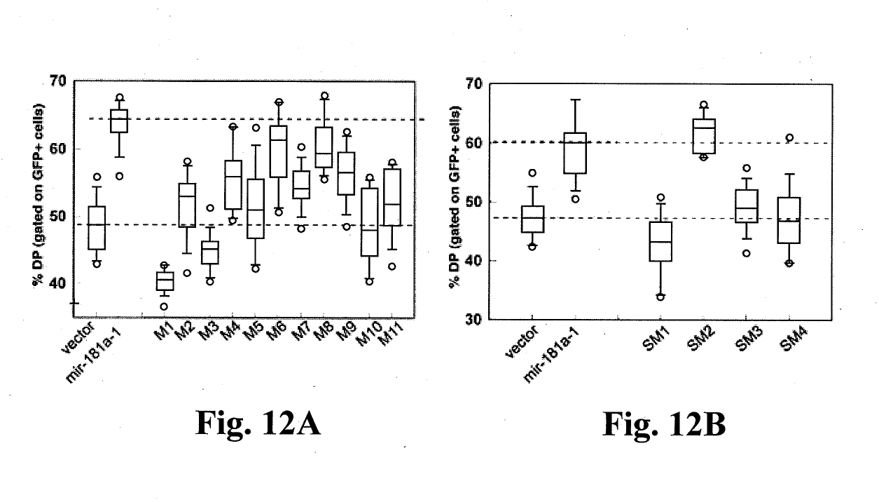

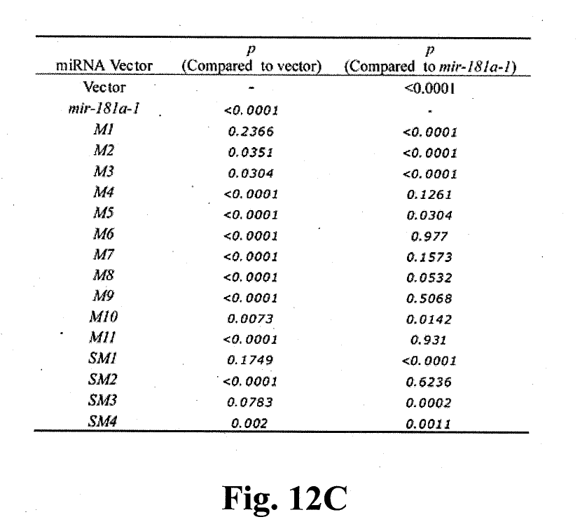

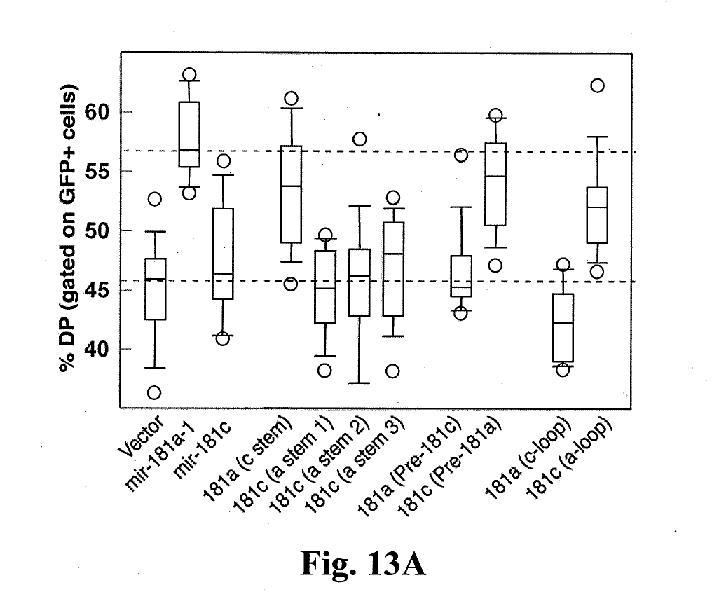

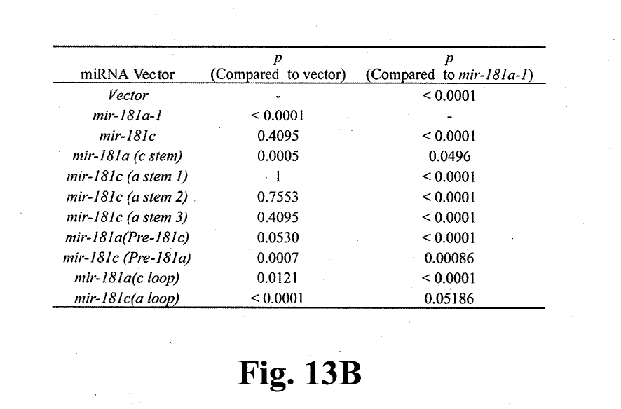

To test this idea, we examined the abilities of mir-181a-1 and mir-181c in promoting DP cell development. Of note, mature miR-181a and miR-181c are differentially expressed during T cell development in the thymus (11, 12) (FIG. 11), indicating that both miRNA genes are processed in thymocytes and may have roles in normal thymocyte development. Thus, by perturbing mir-181a-1 and mir-181c expression in thymocytes we can interrogate their functions in an RNA milieu that consists of physiologically relevant miRNAs and target mRNAs. Interestingly, while the ectopic expression of mir-181a-1 results in a substantial increase in the generation of DP cells, the expression of mir-181c does not (FIG. 2C, grey), demonstrating that mir-181a-1 but not mir-181c can promote DP cell development.

miRNA Genes Encoding Identical Mature miRNAs can have Distinct Biological Activities

To examine whether the single nucleotide variation in the mature miRNA regions may contribute to their differences in activity, we swapped the stem regions (miR and miR* duplexes) between mir-181a-1 and mir-181c (FIG. 2B, yellow). The resulting “mature chimeric” miRNA genes, termed mir-181a (c stem) and mir-181c (a stem 1), express mature miR-181c and mature miR-181a, respectively. Two additional “mature chimeric” miRNA genes, mir-181c (a stem 2) and mir-181c (a stem 3), were also generated by replacing mature miR-181c with mature miR-181a while maintaining the miR-181c* strand. Even though mir-181a (c-stem) produces mature miR-181c, we observed that this “mature chimeric” miRNA gene was still functionally active in promoting DP cell development, albeit with a median activity of ˜73% that of the wild-type mir-181a-1 (FIG. 2C, yellow). In contrast, the mir-181c (a stem 1, 2, 3) genes, which encode mature miR-181a, have a median activity of ˜21%, 1.7, 9.6% that of the wild-type mir-181a-1, respectively (FIG. 2C, yellow). These results demonstrate that the distinct activities of mir-181a-1 and mir-181c are not caused by the single nucleotide difference between their mature miRNA forms. Most notably, we have proven that miRNA genes encoding identical mature miRNAs, such as mir-181c and mir-181a (c-stem) that encode miR-181c, or mir-181c and mir-181a (c-stem) that encode miR-181a, can have distinct biological activities.

Pre-miRNAs and their Loops Determine the Activities and Specificities of the mir-181 Genes

Since mir-181a-1 and mir-181c have divergent pre-miRNA flanking and loop sequences, we then tested whether their differences in activity are determined by their unique pre-miRNAs or pre-miRNA flanking sequences (FIG. 2B). We generated “pre-miRNA chimeric” genes by swapping the pre-miRNA regions between mir-181a-1 and mir-181c (FIG. 2B, orange). The resulting “pre-miRNA chimeric” genes, termed mir-181a (pre-181c) and mir-181c (pre-181a), encode mature miR-181c and miR-181a, respectively (FIG. 2B). When tested in the OP9-DPL1 co-culture assay, the miRNA gene with pre-miR-181a, mir-181c (pre-181a), can promote DP cell development, albeit with a median activity of ˜52% that of the wild-type mir-181a-1, whereas the miRNA gene with pre-miR-181a, mir-181a (pre-181c), has no activity (FIG. 2C, SI Table 2). These results demonstrate that pre-miRNAs have a key role in determining the distinct biological activities of the mir-181a-1 and mir-181c genes. However, pre-miRNA flanking sequences may also contribute to the functions of the mir-181a-1 and mir-181c genes, since mir-181c (pre-181a) has a reduced activity when compared to the wild-type mir-181a-1.

Since pre-miR-181a-1 and pre-miR-181c differ mainly in their pre-miRNA loop nucleotides (FIG. 2B), we next asked whether pre-miRNA loop sequences are involved in the distinct functions of these two miRNA genes. “Chimeric loop” miRNA genes, mir-181a (c-loop) and mir-181c (a-loop), were generated by swapping the pre-miRNA loops between mir-181a-1 and mir-181c (FIG. 2B, blue). When tested in the OP9-DPL1 co-culture assay, mir-181c (a-loop) can promote DP cell development with a median activity of ˜67% that of wild-type mir-181a-1, while mir-181a (c-loop) is inactive in promoting DP cell development (FIG. 2C, blue), demonstrating that the distinct biological activities of the mir-181a-1 and mir-181c genes are largely determined by the differences in their pre-miRNA loops.

The mir-181a-1 Activity is Sensitive to Nucleotide Changes in its Pre-miRNA Loop

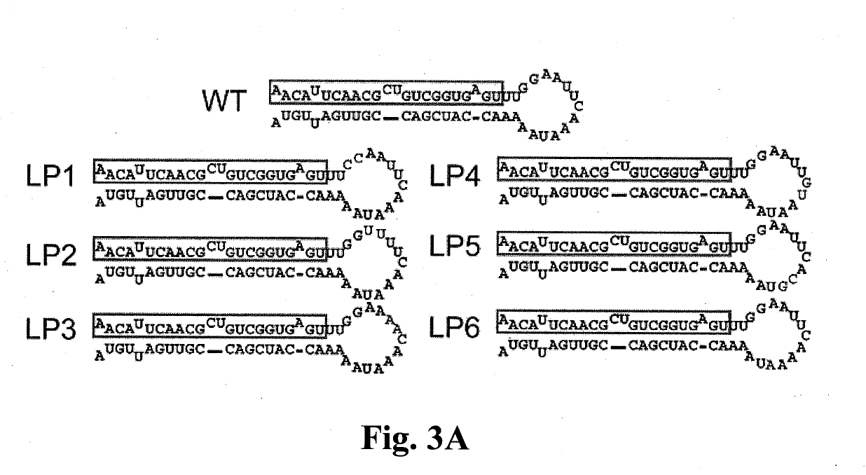

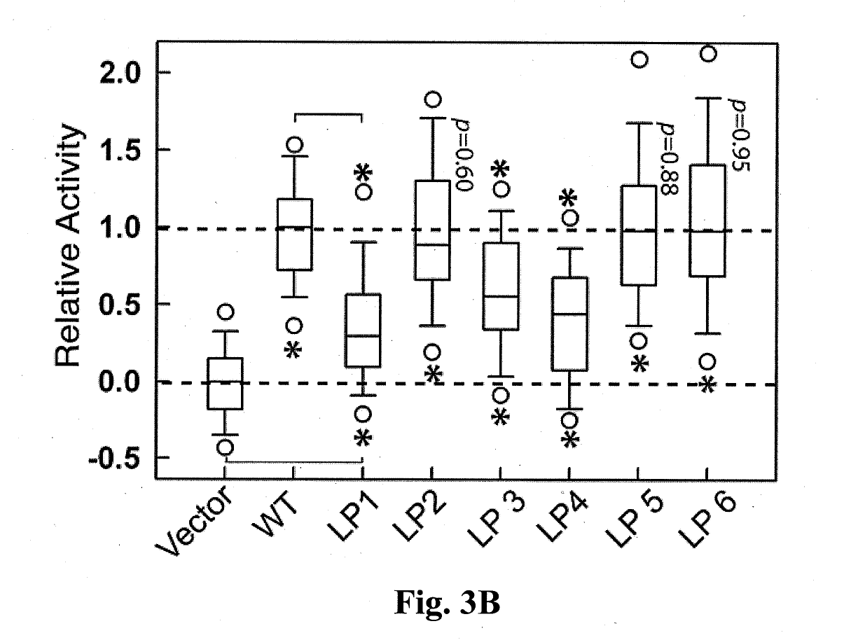

To further investigate the role of pre-miRNA loop nucleotides, we carried out scanning mutagenesis around the pre-miR-181a-1 loop (FIG. 3A). When tested in the OP9-DPL1 co-culture assay, we found that the dinucleotide mutations in the pre-miR-181a-1 loop had varied effects on mir-181a-J activity (FIG. 3B). The LP1, LP3, and LP4 mutants have median activities of ˜29%, 55%, and 46% that of the wild-type mir-181a-1, respectively (FIG. 3B). In contrast, the LP2, LP5 and LP6 mutations did not significantly affect mir-181a-1 activity. The LP1 nucleotides are conserved between the pre-miR-181a and pre-miR-181c loops, but the LP3 and LP4 nucleotides are not, suggesting that LP1 mutations may not contribute to the functional differences between the mir-181a-1 and mir-181c genes. This loop mutagenesis analyses further demonstrate that pre-miRNA loop nucleotides can quantitatively influence the activity of the mir-181a-1 gene.

The Effects of mir-181a-1/c Mutations on Mature miRNA Biogenesis

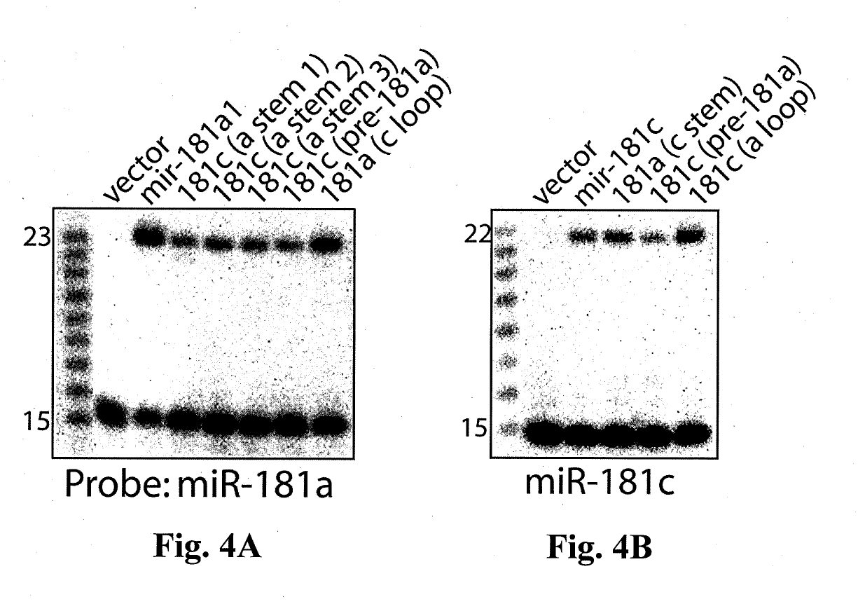

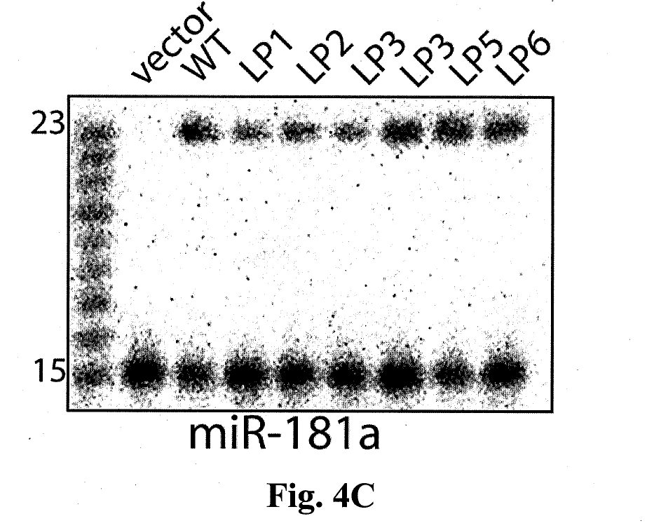

To understand how pre-miRNA loop nucleotides control the activities and specificities of miRNA genes, we characterized the 5′ ends of mature miR-181a and miR-181c produced from various mir-181a-1/c mutants. It is likely that shifts in the 5′ end of mature miRNAs may change the seed nucleotides and affect the activities of the corresponding miRNA genes. To this end we carried out primer extension analyses and showed that mature miRNAs produced from various mir-181a-1/c mutants have the same 5′ end as those produced from the corresponding wild-type mir-181a-1/c genes (FIGS. 4A-C). These results demonstrate that 181a-1/c mutants do not cause changes in 5′ end of the mature miRNA sequences, eliminating the possibility that mir-181a-1/c mutants affect the activities of the mir-181a-1 or mir-181c genes by controlling the fidelity of the 5′ ends of the mature miRNAs produced.

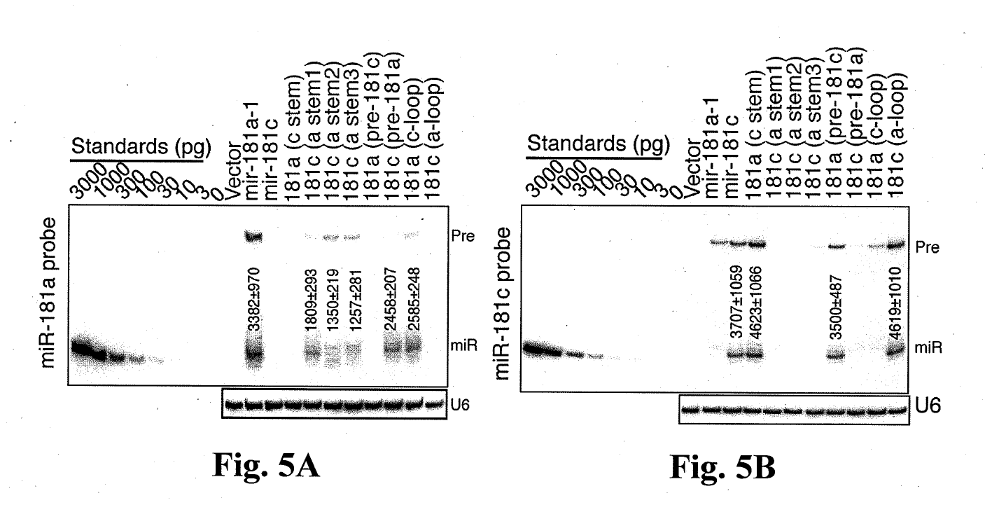

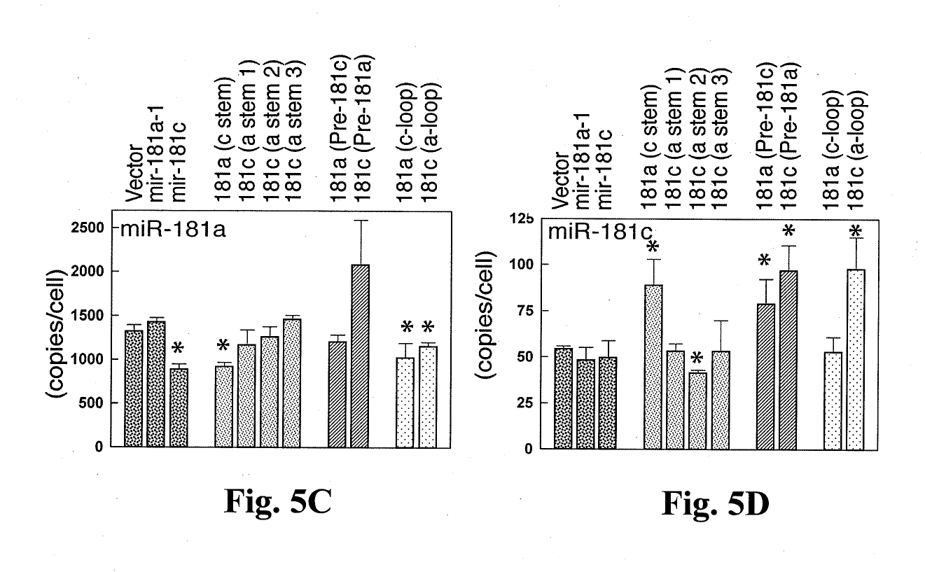

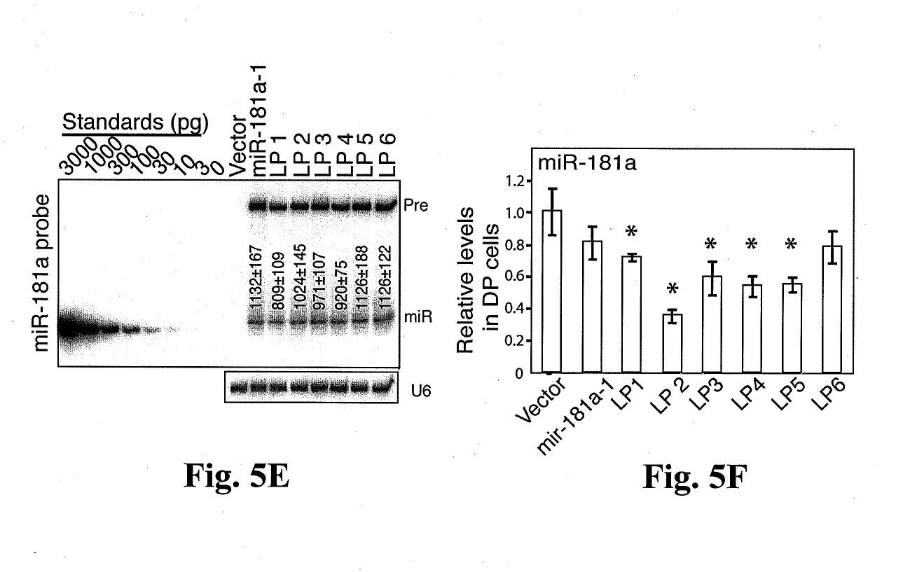

We then investigated whether mir-181a-1/c mutants cause changes in the levels of mature miRNAs made, and if so whether these changes correlate with the activities of corresponding miRNA genes. Quantitative Northern blot analyses were used to define the levels of mature miR-181a and miR-181c, as well as the sizes of the mature miRNAs and the levels of the pre-miRNAs, produced from various mir-181a-1/c mutant constructs. Since it is difficult to obtain sufficient numbers of infected DP thymocytes for Northern blot analyses, we first examined miRNA expression of various mir-181a-1/c mutants in BOSC 23 cells using quantitative Northern blot analyses (FIGS. 5A, B, and 15). In addition, BOSC 23 cells do not express endogenous mature miR-181a or miR-181c, thus allowing for accurate measurement of the levels of mature miRNAs produced. In parallel, we also carried out miRNA qPCR analyses to determine the number of copies of mature miR-181a and miR-181c in DP cells transduced with mir-181a-1/c mutant viruses (FIGS. 5C, 5D). Reasonable quantity of DP cells can be isolated from OP9-DL1 culture by FACS-sorting for miRNA qPCR analysis.

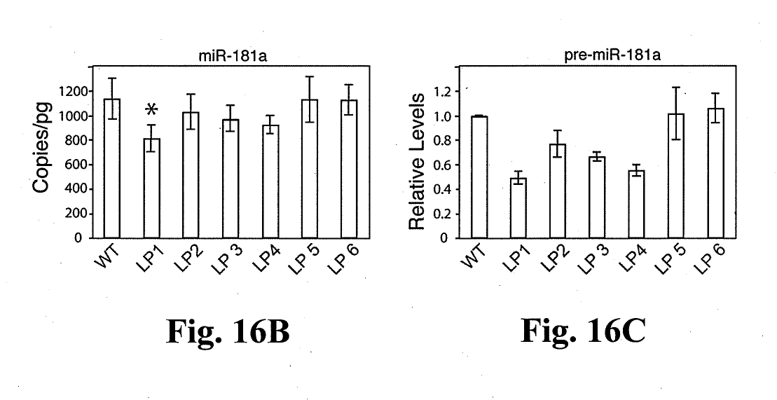

The levels of mature miR-181a in DP cells expressing mir-181a-1/c mutants seem to vary irregularly and have no apparent correlation with the gene activity (FIG. 5C). mir-181a-1 and mir-181c produce comparable levels of mature miR-181a and miR-181c in BOSC 23 cells (FIGS. 5A, B, and 15). However, the ectopic expression of mir-181a-1 or mir-181c does not result in a significant increase in mature miR-181a or miR-181c expression in the infected DP cells (FIGS. 5C, 5D). Ectopic expression of the mir-181c gene actually caused a decrease in the level of mature miR-181a (FIG. 5C), although no decrease in the percentage of DP cells was observed (FIG. 2C, grey). Further, the LP2 mutant exhibits the same activity as wild-type mir-181a-1, but DP cells infected with this mutant expresses ˜50% less mature miR-181a (FIGS. 3B, 5E, 5F). Finally, mature miR-181a levels in DP and BOSC 23 cells expressing the mir-181a-1 loop mutants have no apparent correlation with the activities of mutant genes (FIGS. 3, 5E, 5F, 16). For example, LP3, LP4, and LP5 produced similar levels of mature miR-181a in DP cells and BOSC 23 cells (FIGS. 5E, 5F, 16), but these mutants had different activities in promoting DP cell development (FIG. 3B). LP5 mutations had no effect on mir-181a-1 activity, while LP3 and LP4 mutations caused ˜45% and 56% reduction in median activity, respectively. Thus, the levels of mature miR-181a in DP cells have little correlation with their activities in DP cell development (FIGS. 2, 5C).

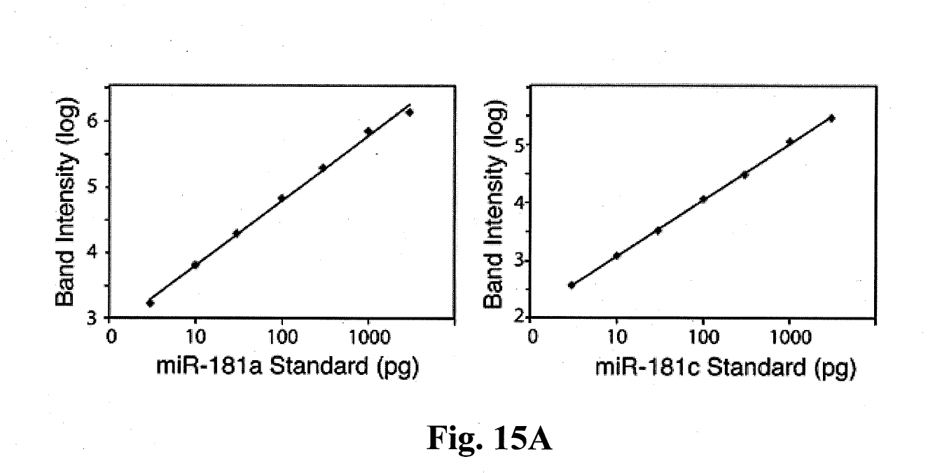

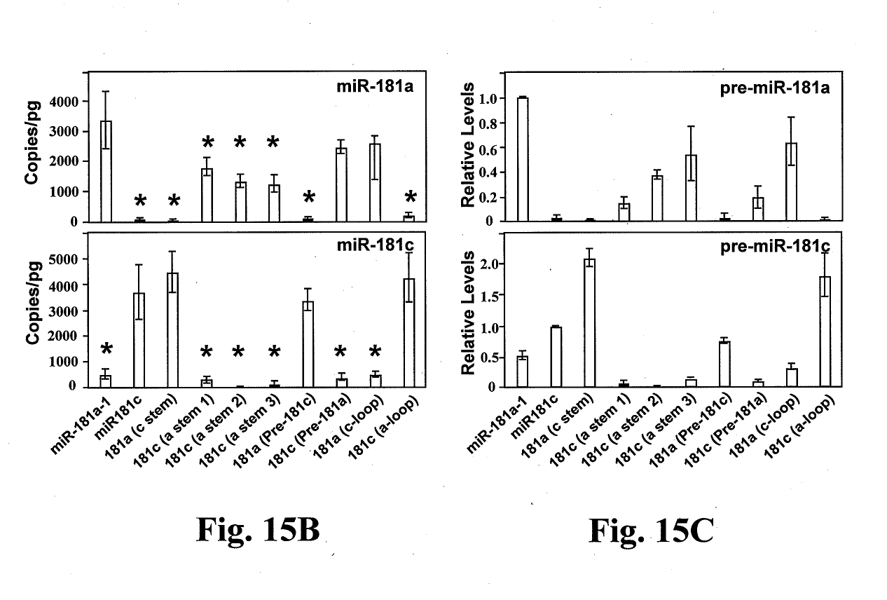

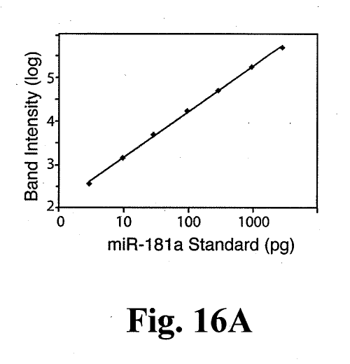

In comparison, the levels of mature miR-181c in DP cells expressing mir-181a-1/c mutants had a partial correlation with their activities in DP cell development, (FIGS. 2, 5D). Ectopic expression of mir-181a (c-stem), mir-181a (pre-181c), and mir-181c α-loop) resulted in significant increases in the levels of mature miR-181c in DP cells (FIG. 5D). However, while mir-181a (c-stem) and mir-181c (a-loop) can promote DP cells development, mir-181a (pre-181c) cannot (FIG. 2C), further showing that increases of mature miR-181c in DP cells do not always correlate with the activities of corresponding miRNA genes. Intriguingly, we noted that mature miRNA expression from some miRNA genes might be differentially regulated in DP and BOSC 23 cells. For example, while mir-181c, mir-181a (c-stem), mir-181a (pre-181c), and mir-181c (a-loop) all produce similar levels of mature miR-181c in BOSC 23 cells (FIGS. 5B and 15), ectopic expression of mir-181a (c-stem), mir-181a (pre-181c), and mir-181c (a-loop), but not mir-181c, resulted in significant increases in the levels of mature miR-181c in DP cells (FIG. 5D). Moreover, among the ones that caused increases in mature miR-181c levels, mir-181a (c-stem) and mir-181c (a-loop) can promote DP cell development, mir-181a (pre-181c) cannot. Overall, mature miR-181a and miR-181c levels in DP cells or BOSC 23 cells expressing the mir-181a-1/c mutants do not consistently correlate with the activities of corresponding miRNA genes (FIGS. 2, 3, 5).

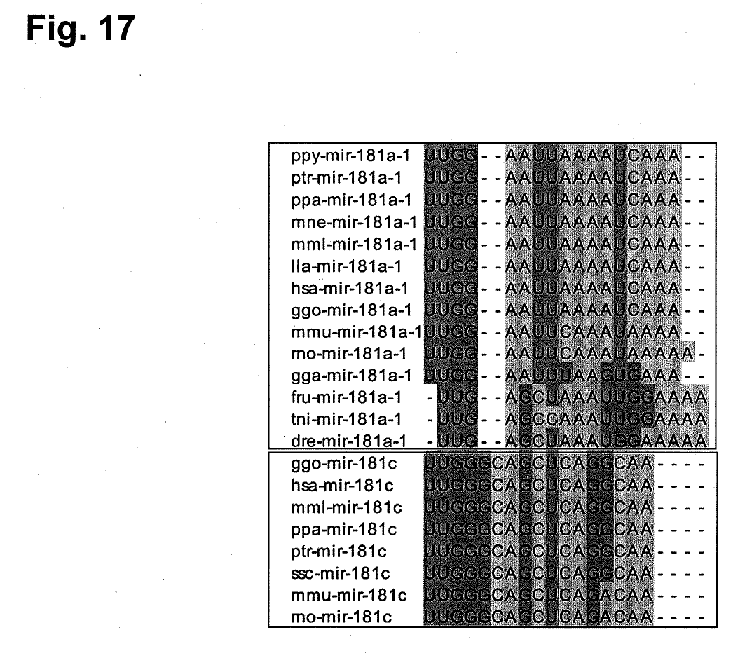

Here have been examined the nucleotide sequences and structural domains that are required for the function of mir-181a-1 and mir-181c through mutagenesis and domain-swapping analyses. We found that both the specificities and activities of mir-181a-1 and mir-181c are controlled by their pre-miRNA loops (FIGS. 2 and 3), and miRNA genes encoding identical or nearly identical mature miRNAs can exert different biological activities determined by their unique loop nucleotides. Interestingly, the pre-miRNA loop sequences of mir-181a-1 and mir-181c are divergent but each is evolutionarily conserved in multiple animal species (FIG. 17), indicating that members of the same miRNA gene families have evolved to achieve distinct specificities or degrees of activity via alterations in their pre-miRNA loop sequences. Intriguingly, mir-181a-1/c mutants do not change the 5′ ends of mature miRNAs produced (FIG. 4) and the levels of mature miRNAs produced from these genes do not always correlate with the activities of corresponding miRNA genes (FIG. 5). These findings together with the fact that pre-miRNA loop nucleotides can control the activities and specificities of mir-181a-1/c genes, demonstrate that mature miRNAs are not the sole determinant for miRNA gene function (FIGS. 2, 3).

Also, pre-miRNA loop nucleotides were thought to have little or no role in either pri-miRNA processing by Drosha or pre-miRNA transport by exportin-5 according to previous biochemical analyses (15-18). However, we have found that mir-181a-1/c mutants affect both mature miRNA and pre-miRNA processing (FIGS. 5A, 5B, 5E, 15 and 16), indicating that pre- and mature miRNA biogenesis is sensitive to pre-miRNA loop nucleotide changes. These findings have revealed a previously unrecognized regulatory complexity of miRNA biogenesis in vivo, suggesting that earlier in vitro analyses on pre- and mature miRNA biogenesis may only in part recapitulate the processing and function of miRNA genes in vivo. When interpreting the above findings, it is critical to draw a distinction between the activity of a miRNA gene and the activity of a mature miRNA. Since long RNA transcripts of the wild-type or mutant miRNA genes were expressed in thymic progenitor cells via retroviral transduction, we have measured the activities of miRNA genes in this study. In contrast, many previous studies probably measured the activities of mature miRNAs by using siRNA duplexes as miRNA surrogates, therefore, did not address the roles of pre-miRNA loop and flanking sequences in miRNA gene function (8, 19).

We were unable to test whether transfected mature miR-181a and miR-181c might be functionally equivalent to full-length mir-181a-1 and mir-181c in the OP9-DL1 co-culture assays. Transfected miRNAs are diluted quickly during cell expansion in a long-term culture assay. Further, complex small RNA sorting pathways may limit efficient and specific incorporation of transfected miRNAs into the pathways used by mir-181a-1/c mutants (20, 21). Also important to note, since multiple small RNAs are made from the primary miRNA transcripts, mutations and deletions in miRNA genes invariably affected the pri-, pre-, and mature miRNA species. Thus, phenotypes observed for mir-181a-1/c mutant genes cannot be attributed to one particular small RNA species in these analyses. Such limitations would also apply to genetic analyses on specific miRNA genes in worms, in which loss-of-function of miRNA genes was only rescued with genomic fragments encoding their pri-miRNAs but not with mature miRNAs (2, 22, 23). In fact, it was recognized that precursor lin-4 RNA contain the mature lin-4 sequence, and genetic analyses were unable to definitively rule out the possible involvement of precursor lin-4 RNA in target gene binding and recognition (2).

Despite that we did not observe consistent correlations between mature miRNA levels and the activities of miRNA genes (FIGS. 2, 3, 5), we could not rule out the possibility that mutations in mir-181a-1/c genes might have affected the activities of mature miRNAs produced from these genes. Likewise, we could not rule out the possibility that pri-miRNAs and pre-miRNAs might have functions independent of the mature miRNAs. Nevertheless, our findings revealed unexpected regulatory complexities encoded in the pre-miRNA loops, suggesting that many scenarios exist by which pre-miRNA loop nucleotides may control the activities of miRNA genes. (FIG. 6). Given that pre-miRNAs contain not only the mature miRNA sequences that can pair with cognate target sites but also the loop nucleotides that are important for the activities and specificities of mir-181a-1 and mir-181c, it would appear that pre-miRNA loops may actually have a functional role in target gene binding and recognition. This model would readily explain why pre-miRNA loops are important for the function of miRNA genes. Supporting this model, pre-miRNA-like stem-loop structures have been shown to be a common module for intermolecular RNA:RNA interactions (24-26).
1. Bushati N, Cohen S M (2007) microRNA Functions. Annu Rev Cell Dev Biol 23:175-205
2. Lee R C, Feinbaum R L, Ambros V (1993) The C. elegans heterochronic gene lin-4 encodes small RNAs with antisense complementarity to lin-14. Cell 75:843-854.
3. Wightman B, Ha I, Ruvkun G (1993) Posttranscriptional regulation of the heterochronic gene lin-14 by lin-4 mediates temporal pattern formation in C. elegans. Cell 75:855-862.
4. Bartel D P (2004) MicroRNAs: genomics, biogenesis, mechanism, and function. Cell 116:281-97.
5. Kim V N (2005) MicroRNA biogenesis: coordinated cropping and dicing. Nat Rev Mol Cell Biol 6:376-85
6. Stark A, Brennecke J, Russell R B, Cohen S M (2003) Identification of Drosophila MicroRNA Targets. PLoS Biol 1:E60.
7. Lewis B P, Shih I H, Jones-Rhoades, M W, Bartel D P, Burge C B (2003) Prediction of mammalian microRNA targets. Cell 115:787-98.
8. Doench J G, Sharp P A (2004) Specificity of microRNA target selection in translational repression. Genes Dev 18:504-11.
9. Altuvia Y, et al. (2005) Clustering and conservation patterns of human microRNAs. Nucleic Acids Res 33:2697-706.
10. Chen C Z, Li L, Lodish H F, Bartel D P (2004) MicroRNAs modulate hematopoietic lineage differentiation. Science 303:83-6.
11. Li Q J, et al. (2007) miR-181a Is an Intrinsic Modulator of T Cell Sensitivity and Selection. Cell 129:147-61.
12. Neilson J R, Zheng G X, Burge C B, Sharp P A (2007) Dynamic regulation of miRNA expression in ordered stages of cellular development. Genes Dev 21:578-89.
13. Zhou B, Wang S, Mayr C, Bartel D P, Lodish, H. F. (2007) miR-150, a microRNA expressed in mature B and T cells, blocks early B cell development when expressed prematurely. Proc Natl Acad Sci USA 104:7080-5.
14. Ciofani M, Knowles G C, Wiest D L, von Boehmer H, Zuniga-Pflucker J C (2006) Stage-specific and differential notch dependency at the alphabeta and gammadelta T lineage bifurcation. Immunity 25:105-16.
15. Lee Y, et al. (2003) The nuclear RNase III Drosha initiates microRNA processing. Nature 425:415-9.
16. Denli, A. M., Tops, B. B., Plasterk, R. H., Ketting, R. F. & Hannon, G. J. (2004) Processing of primary microRNAs by the Microprocessor complex. Nature 432:231-5.
17. Han J, et al. (2006) Molecular basis for the recognition of primary microRNAs by the Drosha-DGCR8 complex. Cell 125:887-901.
18. Yi R, Qin Y, Macara I G, Cullen B R (2003) Exportin-5 mediates the nuclear export of pre-microRNAs and short hairpin RNAs. Genes Dev 17:3011-6.
19. Lim L P et al. (2005) Microarray analysis shows that some microRNAs downregulate large numbers of target mRNAs. Nature 433:769-73.
20. Steiner, F A, et al. (2007) Structural features of small RNA precursors determine Argonaute loading in Caenorhabditis elegans. Nat Struct Mol Biol 14:927-33.
21. Forstemann K, Horwich M D, Wee L, Tomari Y, Zamore P D (2007) Drosophila microRNAs Are Sorted into Functionally Distinct Argonaute Complexes after Production by Dicer-1. Cell 130:287-97.
22. Reinhart B J et al. (2000) The 21 nucleotide let-7 RNA regulates developmental timing in Caenorhabditis elegans. Nature 403:901-906.
23. Johnston R J, Hobert O (2003) A microRNA controlling left/right neuronal asymmetry in Caenorhabditis elegans. Nature 426:845-9.
24. Eisinger J, Gross N (1974) The anticodon-anticodon complex. J Mol Biol 88:165-74.
25. Tomizawa J (1984) Control of ColE1 plasmid replication: the process of binding of RNA I to the primer transcript. Cell 38:861-70.
26. Eguchi Y, Itoh T, Tomizawa J (1991) Antisense RNA. Annu Rev Biochem 60:631-52.

### Methods

Retroviral Constructs for miRNA Gene Expression

A double-copy retroviral vector with a human H1 polymerase III expression cassette was used to express mir-181a-1, mir-181c, and their mutant genes (10, 11). A GFP reporter driven by an independent murine 3-phosphoglycerate kinase promoter (P_{PGK}) was used as a marker for infection.

### OP9-DL1 Stromal Co-Culture Assay for In Vitro T Cell Differentiation

An OP9-DL1 stromal co-culture assay was used for measuring the effects of the mir-181a genes on DP cell development in vitro (see below for details). We use box-plots to summarize the distribution of relative miRNA activity in DP cell development. The ends of the boxes define the 25^{th} and 75^{th} percentiles, a line indicates the median, and bars define the 5^{th} and 95^{th} percentiles. Individual outliers are also shown. The activities of mir-181a-1, mir-181c, and mutant genes in DP cell development were normalized so that the empty vector (negative control) had a median activity of “0” and the mir-181a-1 expressing vector (positive control) had a median activity of “1.” Since limited progenitor cells can be isolated from each mouse, it is often not possible to analyze all miRNA mutant constructs in a single T cell assay; thus, such normalization allows comparison between different experimental data sets. Mann-Whitney Rank Sum Tests were performed to determine whether the activities of mutants were statistically different from the negative control or mir-181a-1 positive control vectors.

miRNA Expression and Processing Analyses

Primer extension, quantitative Northern blot, and TaqMan miRNA qPCR analyses were all used to determine the expression and processing of pre-miRNAs and mature miRNAs from various miRNA genes (see below for details).

Retroviral Constructs for miRNA Gene Expression

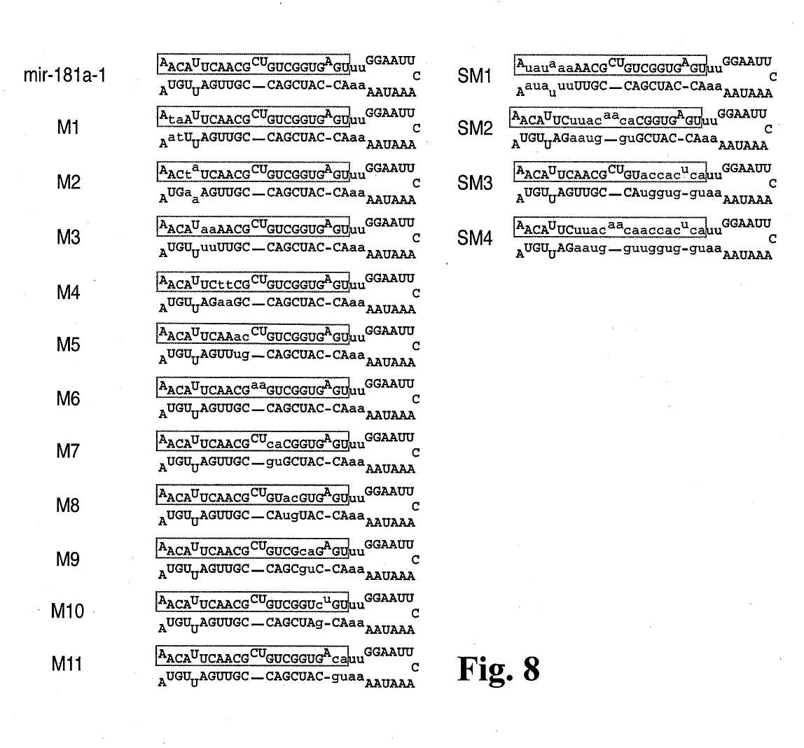

A double-copy retroviral vector with a human H1 polymerase III expression cassette was used to express mir-181a-1, mir-181c, and their mutant genes. Briefly, a ˜270-nt gene segment containing a ˜22-nt mature miRNA and ˜125 nt of genomic sequences flanking both sides of the miRNA was amplified from genomic DNA and placed in the U3 region of the 3′ LTR under the control of the human H1 pol III promoter (a, b). A GFP reporter driven by an independent murine 3-phosphoglycerate kinase promoter (P_{PGK}) was used as a marker for infection. mir-181a-1 and mir-181c mutant constructs were generated using an overlapping PCR strategy to introduce mutations in the stem and loop regions of the miRNA genes. All mutant constructs were validated by DNA sequencing. For mutations in the miRNA stem regions, compensatory mutations were also introduced to the miR* strands to preserve the integrity of the stem and loop structures (FIG. 8). High titer retroviral supernatant was generated by co-transfecting the miRNA expression vector and pCLeco packaging construct into BOSC23 cells (293T based viral packaging cell line).

### OP9-DL1 Stromal Co-Culture Assay for In Vitro T Cell Differentiation.

Six-week old male C57BL/6J mice were obtained from the Jackson Laboratory (Bar Harbor, Me.). Mice were administered a single intravenous dose of 5-fluorouracil (5-FU; 150 mg/kg body weight; SIGMA, St. Louis, Mo.) 4 days before culture initiation. Animals were treated in accordance with Stanford University and Administrative Panels on Laboratory Animal Care guidelines. Thymocytes were isolated from the 5-FU (Fluorouracil) primed-mice, infected with miRNA expression vectors by spinoculation, and seeded at 1×10⁵ infected cells/well into 24-well tissue culture plates containing a monolayer of OP9-DL1 stromal cells. For each viral construct, 12 independent culture replicates were seeded. The cells were cultured in Minimum Essential Medium (MEM) Alpha Medium supplemented with 20% FCS, 10 mM Hepes, 1 mM Sodium pyruvate, 5 ng/ml IL-7, and 27.5 ng/ml Flk2/Flt3L for 24 hours and then medium was changed to remove non-adherent thymocytes. The cultures were fed with fresh medium on day 6. After about 8-10 days of culturing, cells were harvested and stained for surface marker CD4, CD8, and CD45. Percentage of DP cells yielded from culture was quantified by flow cytometry. Both adherent and non-adherent cells were collected. Adherent cells were removed by treatment with collagenase type VI (0.8 mg/ml; Worthington, Lakewood, N.J.) followed by forceful pipetting. Cells were then immunolabeled with PE-conjugated anti-CD4 antibody (clone RM4-5; BD Pharmingen, San Diego, Calif.) and PE-Cy5-conjugated anti-CD8a antibody (clone 53-6.7; BD Pharmingen) and analyzed on a FACSCalibur (BD Biosciences, San Jose, Calif.) for the expression of CD4 and CD8 cell surface antigens. GFP positive thymocytes were distinguishable from GFP positive stromal cells by FSC/SSC gate and the intensity of green fluorescence. When the infect-rate was low, anti-CD45 antibody staining was used to gate out contaminating GFP+OP9-DL1 cells. The appropriate dilution for each antibody was determined prior to use.

Box-plots summarize the distribution of relative miRNA activity in DP cell development. The ends of the boxes define the 25^{th} and 75^{th} percentiles, a line indicates the median, and bars define the 5^{th} and 95^{th} percentiles. Individual outliers are also shown. The activities of mir-181a-1, mir-181c, and mutant genes in DP cell development were normalized so that the empty vector (negative control) has a median activity of “0” and mir-181a-1 expressing vector (positive control) had a median activity of “1.” The percentage of DP cells yielded from the co-culture assay varies between experiments possibly due to heterogeneous nature of the thymic progenitor cells and intrinsic variation between the batches of mice used. Therefore, such normalization is necessary to reset the baseline and allows for comparison among the independent repeats. Mann-Whitney Rank Sum Tests were performed to determine whether the activities of individual 2-nt mutants were statistically different from the control vector or the mir-181a-1 vector.

### Quantitative Northern Blot Analyses

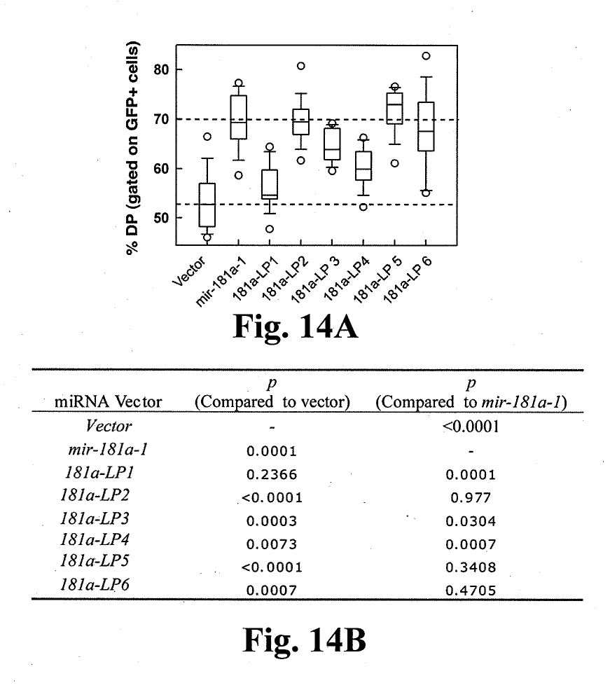

Quantitative Northern blot analyses were used to determine the level of mature miRNA expression and processing of the pre-miRNA and mature miRNA. Total RNA was prepared from BOSC 23 cells transfected with constructs expressing mir-181a-1, or mir-181c, or their mutant genes and loaded onto 15% PAGE gel (10 μg/sample). Since all of the miRNA expressing vectors contain an independent GFP reporter, percentage cells that are GFP positive were determined by FACS analyses and used to control for variations in transfection efficiency. Various amounts of synthetic mature miRNA were loaded onto the same gel to generate standard curves. Specific probes that perfectly match to mature miRNAs were used in hybridization to determine the expression of both mature and pre-miRNA species. Band intensity was determined by phosphoimager quantification. Blots were also probed with U6 probes for normalizing loading. Exact copies/pg of total RNA were determined by comparing to the corresponding standard curve. Representative blots of four or more independent quantitative Northern blot analyses were shown in FIG. 5. Standard Curves and average results of four independent quantitative Northern blot analyses were summarized and plotted (FIGS. 14 and 15).

DNA Oligos were Used as Northern Blot Probes.

Following are the Northern Probe sequences for miR-181a, miR-181c, and stem mutants.

| | |
|---|---|
| miR-181a | |
| ACTCACCGACAGCGTTGAATGTT | (SEQ ID NO:26) |
| miR-181c | |
| ACTCACCGACAG GTTGAATGTT | (SEQ ID NO:27) |
| Ml (613, #831) | |
| TCACCGACAGCGTTGAATTAT | (SEQ ID NO:28) |
| M2 (612, #830) | |
| TCACCGACAGCGTTGATAGTT | (SEQ ID NO:29) |
| M3 (611, #829) | |
| TCACCGACAGCGTTTTATGTT | (SEQ ID NO:30) |
| M4 (610, #828) | |
| TCACCGACAGCGAAGAATGTT | (SEQ ID NO:31) |
| M5 (638, #898) | |
| ACTCACCGACAGGTTTGAATGTT | (SEQ ID NO:32) |
| M6 (608, #826) | |
| TCACCGACTTCGTTGAATGTT | (SEQ ID NO:33) |
| M7 (637, #897) | |
| ACTCACCGTGAGCGTTGAATGTT | (SEQ ID NO:34) |
| M8 (636, #896) | |
| ACTCACGTACAGCGTTGAATGTT | (SEQ ID NO:35) |
| M9 (635, #895) | |
| ACTCTGCGACAGCGTTGAATGTT | (SEQ ID NO:36) |
| M10 (639, #899) | |
| ACAGACCGACAGCGTTGAATGTT | (SEQ ID NO:37) |
| M11 (640, #900) | |
| TGTCACCGACAGCGTTGAATGTT | (SEQ ID NO:38) |
| SM1 (644, #904) | |
| ACTCACCGACAGCGTTTTTATAT | (SEQ ID NO:39) |
| SM2 (641, #901) | |
| TGAGTGGTACAGCGTTGAATGTT | (SEQ ID NO:40) |
| SM3 (642, #902) | |
| ACTCACCGTGTTGTAAGAATGTT | (SEQ ID NO:41) |
| SM4 (643, #903) | |
| TGAGTGGTTGTTGTAAGAATGTT | (SEQ ID NO:42) |

### Primer Extension Analyses

Primer extension was used to map the 5′ ends of the mature miRNAs produced from the mir-181a-1/c mutant genes. Total RNA was prepared from BOSC23 cells 48 hours after transfection with constructs expressing mir-181a-1, or mir-181c, or their mutant genes. P³² labeled primer was mixed with appropriate RNA samples (10 ug total RNA) in the reaction buffer (1×RT reaction buffer with 0.25 mM of each dNTP), heated at 55° C. for 20 minutes, and slowly cooled to 16° C. to allow for annealing. The primer extension reaction was initiated by adding reverse transcriptase at 16° C. for 20 minutes, 42° C. for 2 hours, 85° C. for 5 minutes. Samples were loaded onto a 15% denaturing PAGE gel. Synthetic miR-181a or miR-181c oligos in single nucleotide increments (15 nt-22/23 nt) were labeled and loaded onto the gel as size ladder. The primer extension results were visualized by overnight exposure to phosphoimager screen.

### The Extension Primers:

| | |
|---|---|
| 15 nt miR-181a primer: | |
| 5′ ACTCACCGACAGCGT 3′ | (SEQ ID NO:43) |
| 15 nt miR-181c primer: | |
| 5′ ACTCACCGACAGCGT 3′ | (SEQ ID NO:44) |

miR-181a Ladder Oligos:

| | |
|---|---|
| 16 nt: | |
| ACTCACCGACAGCGtT | (SEQ ID NO:45) |
| 17 nt: | |
| ACTCACCGACAGCGtTG | (SEQ ID NO:46) |
| 18 nt: | |
| ACTCACCGACAGCGtTGA | (SEQ ID NO:47) |
| 19 nt: | |
| ACTCACCGACAGCGtTGAA | (SEQ ID NO:48) |
| 20 nt: | |
| ACTCACCGACAGCGtTGAAT | (SEQ ID NO:49) |
| 21 nt: | |
| ACTCACCGACAGCGtTGAATG | (SEQ ID NO:50) |
| 22 nt: | |
| ACTCACCGACAGCGtTGAATGT | (SEQ ID NO:51) |
| 23 nt: | |
| ACTCACCGACAGCGtTGAATGTT | (SEQ ID NO:52) |

miR-181c Ladder Oligos:

| | |
|---|---|
| 16 nt: | |
| ACTCACCGACAGGTTG | (SEQ ID NO:53) |
| 17 nt: | |
| ACTCACCGACAGGTTGA | (SEQ ID NO:54) |
| 18 nt: | |
| ACTCACCGACAGGTTGAA | (SEQ ID NO:55) |
| 19 nt: | |
| ACTCACCGACAGGTTGAAT | (SEQ ID NO:56) |
| 20 nt: | |
| ACTCACCGACAGGTTGAATG | (SEQ ID NO:57) |
| 21 nt: | |
| ACTCACCGACAGGTTGAATGT | (SEQ ID NO:58) |
| 22 nt: | |
| ACTCACCGACAGGTTGAATGTT | (SEQ ID NO:59) |

miRNA qPCR Analyses

GFP positive DP T cells from OP9-DL1 co-culture assay were isolated by FACS-sorting (>94% pure). Synthetic miR-223 was spiked into sorted cells at the ratio of 100 pmol of miR-223 per 100,000 cells before RNA purification. Total RNA was isolated using Trizol reagent (Invitrogen, Carlsbad, Calif.). We assumed that the ratio of spiked miR-223 to a miRNA of interest would not change during RNA purification. cDNA was then synthesized using miRNA-specific looped primers (Applied Biosystems, Foster city, CA) and amplified with miRNA specific forward primers, TaqMan probe, and reverse primers (Applied Biosystems). PCR amplification was performed in triplicate in an ABI-7000 sequence detection system (Applied Biosystems) at 95° C. for 10 min followed by 40 cycles at 95° C. for 15 sec and 60° C. for 1 min. To determine exact copy number of a miRNA in sorted DP cells, we carried out absolute miRNA quantification with miRNA qPCR assay. Exact copies of test and spiked miRNAs in defined amount of total RNA input were determined by using standard curves for mature miR-181a, miR-181c, and spiked miR-223. miR-181a or miR-181c expression was normalized using miR-15b as internal loading control. Representative results of three miRNA qPCR analyses of independently sorted virally infected DP cells were shown. All reactions were carried out according to the manufacturer's instructions.
a. Chen, C. Z., L. Li, H. F. Lodish, and D. P. Bartel. 2004. MicroRNAs modulate hematopoietic lineage differentiation. Science 303:83.
b. Li, Q. J., J. Chau, P. J. Ebert, G. Sylvester, H. Min, G. Liu, R. Braich, M. Manoharan, J. Soutschek, P. Skare, L. O. Klein, M. M. Davis, and C. Z. Chen. 2007. miR-181a is an Intrinsic Modulator of T Cell Sensitivity and Selection. Cell 129:147.

It is evident from the above results that the subject invention provides for better and more specific use of RNAi using pre-miRNA where both the seed sequence and the essential nucleotides of the loop sequence provide for specificity in giving unique results not available from the mature stem sequence. Different isoforms of pre-miRNA provide for different phenotypic outcomes, so that by defining both the stem seed sequence and the essential nucleotides of the loop sequence one can specifically control the protein whose activity is being modulated, usually suppressed. By employing both the seed sequence and loop sequence for screening, one can identify mRNAs for which the particular pre-miRNA is specific and modulate its expression in host cells. The subject pre-miRNAs and constructs employing the sequences can be used to express the RNA product in cells under various conditions allowing for investigating cell properties, cell pathways, response of cells and tissue to an external environment, and the like. In addition, one can prepare novel pre-miRNAs with enhanced specificity for a target mRNA, so as to reduce the variations in activity and phenotype due to the multiplicity of mRNAs that are targeted. By controlling the seed sequence and the relevant nucleotides in the loop, pre-miRNAs are produced that are complementary to wild-type miRNAs and can be used in screening of cells for the wild-type miRNAs. The non-wild-type miRNAs can be used to identify target genes, elucidate cellular pathways, determine whether single or multiple mRNAs are modulated and lead to therapeutic miRNAs.

To identify precursor miRNAs as to their capability to regulate expression of one or more genes, one can randomly mutate by one or two nucleotides at least the loop region of wild-type pre-miRNA of precursor genes to produce a family of isoforms of precursor miRNA genes. One can then introduce into cells individual modified family members under the transcriptional control of a regulatory region functional in the cells under conditions providing for induced or constitutive transcription. One can then evaluate any changes in the phenotype of the cell. Phenotype can be measured as to the expression of proteins, differentiation, growth, change in cell type, etc. By using different cell types, one can screen for the effect on phenotype. In those cells that demonstrate a change in phenotype, one can then screen for the mRNAs that bind the active isoforms.

One can also use the random isoform set to screen for complementary sequences in mRNAs. As discussed previously, one can determine the binding of members of a mixture of mRNAs, e.g. a transcriptome. Where a plurality of mRNAs bind, one can determine the affinity of individual mRNAs for individual precursor miRNAs by measuring the level of binding by conventional assays for double stranded RNA, replacement and isolation of the bound mRNAs using sequences complementary to the miRNAs and the measuring the mRNA by conventional procedures, e.g. mass spectrometry.

All references referred to in the text are incorporated herein by reference as if fully set forth herein. All procedures disclosed in the references are incorporated as demonstrating the level of skill in the art to perform the procedures indicated in this application. The relevant portions associated with this document will be evident to those of skill in the art. Any discrepancies between this application and such reference will be resolved in favor of the view set forth in this application.

Although the invention has been described with reference to the above examples, it will be understood that modifications and variations are encompassed within the spirit and scope of the invention. Accordingly, the invention is limited only by the following claims.

What is claimed is:

1. In a method for predicting an mRNA sequence complementary to at least the seed sequence of a stem sequence of an miRNA, wherein said miRNA is produced from a pre-miRNA having a stem sequence and a loop sequence, wherein a seed sequence of from about 5 to 10 nucleotides of a strand of said stem is substantially complementary to a sequence of said mRNA, the improvement which comprises:
including for complementation of a contiguous sequence of at least 2 nucleotides of said mRNA, a sequence of at least 2 nucleotides of said loop sequence, wherein said mRNA sequences complementary to said seed sequence and said 2 nucleotides are separated by a linking group.

2. A method according to claim 1, wherein at least one of said 2 nucleotides is at the 5′ terminus of said loop and at least one other nucleotide is equally spaced in said loop and said mRNA.

3. A method according to claim 1, wherein said seed sequence comprises from 6 to 8 nucleotides.

4. A method for selecting a composition for modulating the expression of a protein from a target mRNA, said method comprising:
selecting from a plurality of nucleic acid sequences that encode for precursor-miRNAs, wherein said precursor miRNAs have a stem and loop sequence with the 5′ sequence having a sequence at least partially complementary to said target mRNA, a sequence that has complementarity of at least 2 nucleotides between the loop sequence and a sequence of said target mRNA, wherein said complementary sequences of said mRNA are within 50 nt.

5. A method according to claim 4, wherein said complementary nucleotides of said loop sequence and said mRNA are equally spaced apart.

6. A mutated precursor miRNA, wherein said precursor miRNA comprises a stem sequence and a loop sequence, wherein said pre-miRNAs and pri-miRNAs have a higher binding affinity and selectivity than the natural pre-miRNA that binds to a target mRNA, and said stem sequence comprises a seed sequence complementary to a sequence present in a target mRNA, said precursor miRNA is mutated by a change in the sequence, wherein the binding affinity is based on both the sequence of said stem and said loop, the terminal nucleotide of said seed sequence being from 16 to 20 nucleotides from the initial 5′ nucleotide of said loop and the complementary sequences of said mRNA to said seed and loop sequences being separated by from about 16 to 20 nucleotides.

7. A mutated precursor miRNA according to claim 6, comprising at least 2 mutations in said loop to provide complementarity between said mutations in said loop and said target mRNA.

8. A mutated precursor miRNA according to claim 6, wherein said loop has at least 4 nucleotides complementary to said target mRNA.

9. A mutated precursor miRNA according to claim 7, wherein said loop sequence has at least 3 nucleotide substitutions that complement said target mRNA.

10. A mutated precursor miRNA according to claim 6, wherein said target mRNA is a target mRNA transcribed in a normal T-cell or a leukemic T cell.

11. A mutated precursor miRNA according to claim 6, wherein at least a portion of a loop sequence of greater than 2 nucleotides from one pre-miRNA is substituted for an equivalent portion of a different pre-miRNA.

12. A mutated precursor miRNA according to claim 11, wherein said one precursor miRNA and said different precursor miRNA are isoforms.

13. A method for modulating the phenotype of a cell, said method comprising:
introducing into a cell a mutated precursor miRNA, wherein said precursor miRNA comprises a stem sequence and a loop sequence, said stem sequence comprising a seed sequence wherein said seed sequence is substantially complementary to a sequence present in a target mRNA produced in said cell, said loop is mutated by substitution of at least one nucleotide to provide at least 4 nucleotides complementary to nucleotides in said mRNA, where the closest nucleotide in said mRNA sequence complementary to a nucleotide in said loop is from about 16 to 20 nucleotides from said mRNA sequence complementary to said seed sequence;
wherein said phenotype of said cell is modulated by the suppression of translation of said mRNA by said mutated precursor miRNA.

14. A method according to claim 13, wherein said at least 4 nucleotides are noncontiguous.

15. A method according to claim 12, wherein said seed sequence has from 6 to 8 nucleotides.

16. A method according to claim 12, wherein said mRNA has a 3′-UTR and said loop sequence has at least 2 nucleotide substitutions that complement said 3′-UTR of said target mRNA.

17. A method according to claim 12, wherein said target mRNA is a target mRNA transcribed in a T-cell.

18. A method according to claim 12, wherein at least a portion of a loop sequence of greater than 2 nucleotides from one precursor miRNA substitutes an equivalent portion of a different precursor miRNA.

19. A method according to claim 18, wherein said one precursor miRNA and said different precursor miRNA are isoforms.

20. A method for identifying the role of a loop sequence of a precursor miRNA among a plurality of isoforms differing in their loop sequences, said method comprising:
substituting at least a portion of one loop from one isoform with an equivalent portion of a loop from a different isoform to produce a mutated precursor miRNA;
introducing said one isoform in a first cell and said mutated precursor miRNA into a second cell;
determining the effect of the one isoform and mutated precursor miRNA on the phenotype of each of said cells;
wherein when said phenotype is the same, said loop sequence affects the role of said precursor miRNA.

21. A method according to claim 20, wherein said cells are T-cells and said phenotype is the differentiation of said T-cells.

22. An antisense nucleic acid sequence complementary to at least a seed sequence and at least the first two nucleotides of a loop sequence of a pre-miRNA.

23. A method for identifying precursor miRNA capable of regulating mRNA translation, said method comprising:
randomly mutating the loop sequence of a precursor miRNA gene by from one to two nucleotides to produce a family of isoforms precursor miRNAs;
introducing a member of said family into a cell under the regulatory control of a transcriptional regulatory region, whereby said member is transcribed;
determining any change in phenotype of said cell as a measure of said member regulating mRNA translation.

24. A method for identifying precursor miRNA capable of binding to candidate mRNAs, said method comprising:
randomly mutating the loop sequence of a precursor miRNA gene by from one to two nucleotides to produce a family of isoforms precursor miRNAs;
combining said family of with a group of mRNAs; and
determining the binding of any members of said family with an mRNA of said group.

25. A method according to claim 24, wherein the level of binding of a binding member of said family is determined as compared to other members of said family.


SEQUENCE LISTING (143 sequences)

SEQ ID NO: 1 - DNA, 6075 bp, Artificial Sequence
  Synthetic polynucleotide
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 2 - DNA, 388 bp, Unknown
  Wild-type mir-181a-1 gene
ctcgagtgtgacaggtttggttaaaggattgggctttcctctgcctccctcctgctccagactcccacagatactgtttaaatcagcacatctctgcctcacaggttgcttcagtgaacattcaacgctgtcggtgagtttggaattcaaataaaaaccatcgaccgttgattgtaccctatagctaaccatcatctactccatggccctctgcgtttgctgaagacagaaccgcaaagcaggacccgacaggattcttttttaattaagaattcctaggaattcttgccaaacctacaggtggggtctttcattcccccctttttctggagactaaataaaatcttttattttatcgataagcttggctgcaggtcgacgcggccgc

SEQ ID NO: 3 - DNA, 60 bp, Unknown
  Wild-type pre-miR-181a-1 precursor
aacattcaacgctgtcggtgagtttggaattcaaataaaaaccatcgaccgttgattgta

SEQ ID NO: 4 - DNA, 60 bp, Artificial Sequence
  Chemically synthesized 2-nt mature miR-181a-1 mutant
ataattcaacgctgtcggtgagtttggaattcaaataaaaaccatcgaccgttgatttaa

SEQ ID NO: 5 - DNA, 60 bp, Artificial Sequence
  Chemically synthesized 2-nt mature miR-181a-1 mutant
aactatcaacgctgtcggtgagtttggaattcaaataaaaaccatcgaccgttgaaagta

SEQ ID NO: 6 - DNA, 60 bp, Artificial Sequence
  Chemically synthesized 2-nt mature miR-181a-1 mutant
aacataaaacgctgtcggtgagtttggaattcaaataaaaaccatcgaccgttttttgta

SEQ ID NO: 7 - DNA, 60 bp, Artificial Sequence
  Chemically synthesized 2-nt mature miR-181a-1 mutant
aacattcttcgctgtcggtgagtttggaattcaaataaaaaccatcgaccgaagattgta

SEQ ID NO: 8 - DNA, 60 bp, Artificial Sequence
  Chemically synthesized 2-nt mature miR-181a-1 mutant
aacattcaaacctgtcggtgagtttggaattcaaataaaaaccatcgacgtttgattgta

SEQ ID NO: 9 - DNA, 60 bp, Artificial Sequence
  Chemically synthesized 2-nt mature miR-181a-1 mutant
aacattcaacgaagtcggtgagtttggaattcaaataaaaaccatcgaccgttgattgta

SEQ ID NO: 10 - DNA, 60 bp, Artificial Sequence
  Chemically synthesized 2-nt mature miR-181a-1 mutant
aacattcaacgctcacggtgagtttggaattcaaataaaaaccatcgtgcgttgattgta

SEQ ID NO: 11 - DNA, 60 bp, Artificial Sequence
  Chemically synthesized 2-nt mature miR-181a-1 mutant
aacattcaacgctgtacgtgagtttggaattcaaataaaaaccatgtaccgttgattgta

SEQ ID NO: 12 - DNA, 60 bp, Artificial Sequence
  Chemically synthesized 2-nt mature miR-181a-1 mutant
aacattcaacgctgtcgcagagtttggaattcaaataaaaacctgcgaccgttgattgta

SEQ ID NO: 13 - DNA, 60 bp, Artificial Sequence
  Chemically synthesized 2-nt mature miR-181a-1 mutant
aacattcaacgctgtcggtctgtttggaattcaaataaaaacgatcgaccgttgattgta

SEQ ID NO: 14 - DNA, 60 bp, Artificial Sequence
  Chemically synthesized 2-nt mature miR-181a-1 mutant
aacattcaacgctgtcggtgacattggaattcaaataaaatgcatcgaccgttgattgta

SEQ ID NO: 15 - DNA, 60 bp, Artificial Sequence
  Chemically synthesized segment mature miR-181a-1 mutant
atataaaaacgctgtcggtgagtttggaattcaaataaaaaccatcgaccgtttttataa

SEQ ID NO: 16 - DNA, 60 bp, Artificial Sequence
  Chemically synthesized segment mature miR-181a-1 mutant
aacattcttacaacacggtgagtttggaattcaaataaaaaccatcgtggtaagattgta

SEQ ID NO: 17 - DNA, 60 bp, Artificial Sequence
  Chemically synthesized segment mature miR-181a-1 mutant
aacattcaacgctgtaccactcattggaattcaaataaaatggtggtaccgttgattgta

SEQ ID NO: 18 - DNA, 60 bp, Artificial Sequence
  Chemically synthesized segment mature miR-181a-1 mutant
aacattcttacaacaaccactcattggaattcaaataaaatggtggttggtaagattgta

SEQ ID NO: 19 - DNA, 60 bp, Artificial Sequence
  Chemically synthesized miR-181a-1 Loop Mutant
aacattcaacgctgtcggtgagtttccaattcaaataaaaaccatcgaccgttgattgta

SEQ ID NO: 20 - DNA, 60 bp, Artificial Sequence
  Chemically synthesized miR-181a-1 Loop Mutant
aacattcaacgctgtcggtgagtttggttttcaaataaaaaccatcgaccgttgattgta

SEQ ID NO: 21 - DNA, 60 bp, Artificial Sequence
  Chemically synthesized miR-181a-1 Loop Mutant
aacattcaacgctgtcggtgagtttggaaaacaaataaaaaccatcgaccgttgattgta

SEQ ID NO: 22 - DNA, 60 bp, Artificial Sequence
  Chemically synthesized miR-181a-1 Loop Mutant
aacattcaacgctgtcggtgagtttggaattgtaataaaaaccatcgaccgttgattgta

SEQ ID NO: 23 - DNA, 60 bp, Artificial Sequence
  Chemically synthesized miR-181a-1 Loop Mutant
aacattcaacgctgtcggtgagtttggaattcacgtaaaaaccatcgaccgttgattgta

SEQ ID NO: 24 - DNA, 60 bp, Artificial Sequence
  Chemically synthesized miR-181a-1 Loop Mutant
aacattcaacgctgtcggtgagtttggaattcaaaataaaaccatcgaccgttgattgta

SEQ ID NO: 25 - DNA, 290 bp, Unknown
  Wild-type mir-181c gene
gatcctcgaggtgagtcaaaggggaccctggtttctctctcgtcccacatgctctctgccttgctggcctactctcccaactccagttatccaagaacttgccaagggtttgggggaacattcaacctgtcggtgagtttgggcagctcagacaaaccatcgaccgttgagtggaccccgaggcctggaactgccacccgtctaccccatccccaccctgtagacccgggagagccccaggcagcatcccctgcctcaggccacagcaaaggtcacaattgaattcgatc

SEQ ID NO: 26 - DNA, 23 bp, Artificial Sequence
  Chemically synthesized Northern Probe sequence
actcaccgacagcgttgaatgtt

SEQ ID NO: 27 - DNA, 22 bp, Artificial Sequence
  Chemically synthesized Northern Probe sequence
actcaccgacaggttgaatgtt

SEQ ID NO: 28 - DNA, 21 bp, Artificial Sequence
  Chemically synthesized Northern Probe sequence
tcaccgacagcgttgaattat

SEQ ID NO: 29 - DNA, 21 bp, Artificial Sequence
  Chemically synthesized Northern Probe sequence
tcaccgacagcgttgatagtt

SEQ ID NO: 30 - DNA, 21 bp, Artificial Sequence
  Chemically synthesized Northern Probe sequence
tcaccgacagcgttttatgtt

SEQ ID NO: 31 - DNA, 21 bp, Artificial Sequence
  Chemically synthesized Northern Probe sequence
tcaccgacagcgaagaatgtt

SEQ ID NO: 32 - DNA, 23 bp, Artificial Sequence
  Chemically synthesized Northern Probe sequence
actcaccgacaggtttgaatgtt

SEQ ID NO: 33 - DNA, 21 bp, Artificial Sequence
  Chemically synthesized Northern Probe sequence
tcaccgacttcgttgaatgtt

SEQ ID NO: 34 - DNA, 23 bp, Artificial Sequence
  Chemically synthesized Northern Probe sequence
actcaccgtgagcgttgaatgtt

SEQ ID NO: 35 - DNA, 23 bp, Artificial Sequence
  Chemically synthesized Northern Probe sequence
actcacgtacagcgttgaatgtt

SEQ ID NO: 36 - DNA, 23 bp, Artificial Sequence
  Chemically synthesized Northern Probe sequence
actctgcgacagcgttgaatgtt

SEQ ID NO: 37 - DNA, 23 bp, Artificial Sequence
  Chemically synthesized Northern Probe sequence
acagaccgacagcgttgaatgtt

SEQ ID NO: 38 - DNA, 23 bp, Artificial Sequence
  Chemically synthesized Northern Probe sequence
tgtcaccgacagcgttgaatgtt

SEQ ID NO: 39 - DNA, 23 bp, Artificial Sequence
  Chemically synthesized Northern Probe sequence
actcaccgacagcgtttttatat

SEQ ID NO: 40 - DNA, 23 bp, Artificial Sequence
  Chemically synthesized Northern Probe sequence
tgagtggtacagcgttgaatgtt

SEQ ID NO: 41 - DNA, 23 bp, Artificial Sequence
  Chemically synthesized Northern Probe sequence
actcaccgtgttgtaagaatgtt

SEQ ID NO: 42 - DNA, 23 bp, Artificial Sequence
  Chemically synthesized Northern Probe sequence
tgagtggttgttgtaagaatgtt

SEQ ID NO: 43 - DNA, 15 bp, Artificial Sequence
  Chemically synthesized 15-nt miR-181a primer
actcaccgacagcgt

SEQ ID NO: 44 - DNA, 15 bp, Artificial Sequence
  Chemically synthesized 15nt miR-181c primer
actcaccgacagcgt

SEQ ID NO: 45 - DNA, 16 bp, Artificial Sequence
  Chemically synthesized miR-181a ladder oligo
actcaccgacagcgtt

SEQ ID NO: 46 - DNA, 17 bp, Artificial Sequence
  Chemically synthesized miR-181a ladder oligo
actcaccgacagcgttg

SEQ ID NO: 47 - DNA, 18 bp, Artificial Sequence
  Chemically synthesized miR-181a ladder oligo
actcaccgacagcgttga

SEQ ID NO: 48 - DNA, 19 bp, Artificial Sequence
  Chemically synthesized miR-181a ladder oligo
actcaccgacagcgttgaa

SEQ ID NO: 49 - DNA, 20 bp, Artificial Sequence
  Chemically synthesized miR-181a ladder oligo
actcaccgacagcgttgaat

SEQ ID NO: 50 - DNA, 21 bp, Artificial Sequence
  Chemically synthesized miR-181a ladder oligo
actcaccgacagcgttgaatg

SEQ ID NO: 51 - DNA, 22 bp, Artificial Sequence
  Chemically synthesized miR-181a ladder oligo
actcaccgacagcgttgaatgt

SEQ ID NO: 52 - DNA, 23 bp, Artificial Sequence
  Chemically synthesized miR-181a ladder oligo
actcaccgacagcgttgaatgtt

SEQ ID NO: 53 - DNA, 16 bp, Artificial Sequence
  Chemically synthesized miR-181c ladder oligo
actcaccgacaggttg

SEQ ID NO: 54 - DNA, 17 bp, Artificial Sequence
  Chemically synthesized miR-181c ladder oligo
actcaccgacaggttga

SEQ ID NO: 55 - DNA, 18 bp, Artificial Sequence
  Chemically synthesized miR-181c ladder oligo
actcaccgacaggttgaa

SEQ ID NO: 56 - DNA, 19 bp, Artificial Sequence
  Chemically synthesized miR-181c ladder oligo
actcaccgacaggttgaat

SEQ ID NO: 57 - DNA, 20 bp, Artificial Sequence
  Chemically synthesized miR-181c ladder oligo
actcaccgacaggttgaatg

SEQ ID NO: 58 - DNA, 21 bp, Artificial Sequence
  Chemically synthesized miR-181c ladder oligo
actcaccgacaggttgaatgt

SEQ ID NO: 59 - DNA, 22 bp, Artificial Sequence
  Chemically synthesized miR-181c ladder oligo
actcaccgacaggttgaatgtt

SEQ ID NO: 60 - RNA, 23 nt, Artificial Sequence
  Chemically synthesized mature miRNA miR-181a
aacauucaacgcugucggugagu

SEQ ID NO: 61 - RNA, 22 nt, Artificial Sequence
  Chemically synthesized mature miRNA miR-181c
aacauucaaccugucggugagu

SEQ ID NO: 62 - RNA, 60 nt, Unknown
  Wild-type mir-181a-1 gene
aacauucaacgcugucggugaguuuggaauucaaauaaaaaccaucgaccguugauugua

SEQ ID NO: 63 - RNA, 59 nt, Unknown
  Wild-type miR-181c
aacauucaaccugucggugaguuugggcagcucagacaaaccaucgaccguugagugga

SEQ ID NO: 64 - RNA, 59 nt, Artificial Sequence
  Chemically synthesized mature miRNA 181a (c stem)
aacauucaaccugucggugaguuuggaauucaaauaaaaaccaucgaccguugagugga

SEQ ID NO: 65 - RNA, 60 nt, Artificial Sequence
  Chemically synthesized mature miRNA 181c (a stem 1)
aacauucaacgcugucggugaguuugggcagcucagacaaaccaucgaccguugauugua

SEQ ID NO: 66 - RNA, 59 nt, Artificial Sequence
  Chemically synthesized mature miRNA 181c (a stem 2)
aacauucaacgcugucggugauuugggcagcucagacaaacaucgacccguugagugga

SEQ ID NO: 67 - RNA, 59 nt, Artificial Sequence
  Chemically synthesized mature miRNA 181c (a stem 3)
aacauucaacgcugucggugaguugggcagcucagacaaccaucgacccguugagugga

SEQ ID NO: 68 - RNA, 60 nt, Artificial Sequence
  Chemically synthesized pre-miR-181a-1 loop mutant LP1
aacauucaacgcugucggugaguuuccaauucaaauaaaaaccaucgaccguugauugua

SEQ ID NO: 69 - RNA, 60 nt, Artificial Sequence
  Chemically synthesized pre-miR-181a-1 loop mutant LP2
aacauucaacgcugucggugaguuugguuuucaaauaaaaaccaucgaccguugauugua

SEQ ID NO: 70 - RNA, 60 nt, Artificial Sequence
  Chemically synthesized pre-miR-181a-1 loop mutant LP3
aacauucaacgcugucggugaguuuggaaaacaaauaaaaaccaucgaccguugauugua

SEQ ID NO: 71 - RNA, 60 nt, Artificial Sequence
  Chemically synthesized pre-miR-181a-1 loop mutant LP4
aacauucaacgcugucggugaguuuggaauuguaauaaaaaccaucgaccguugauugua

SEQ ID NO: 72 - RNA, 60 nt, Artificial Sequence
  Chemically synthesized pre-miR-181a-1 loop mutant LP5
aacauucaacgcugucggugaguuuggaauucacguaaaaaccaucgaccguugauugua

SEQ ID NO: 73 - RNA, 60 nt, Artificial Sequence
  Chemically synthesized pre-miR-181a-1 loop mutant LP6
aacauucaacgcugucggugaguuuggaauucaaaauaaaaccaucgaccguugauugua

SEQ ID NO: 74 - RNA, 23 nt, Artificial Sequence
  Synthetic oligonucleotide
aacauucaacgcugucggugagu

SEQ ID NO: 75 - RNA, 17 nt, Artificial Sequence
  Synthetic oligonucleotide
uuggaauucaaauaaaa

SEQ ID NO: 76 - RNA, 23 nt, Artificial Sequence
  Chemically synthesized mir-181a-1mutant M1
auaauucaacgcugucggugagu

SEQ ID NO: 77 - RNA, 23 nt, Artificial Sequence
  Chemically synthesized mir-181a-1mutant M2
aacuaucaacgcugucggugagu

SEQ ID NO: 78 - RNA, 23 nt, Artificial Sequence
  Chemically synthesized mir-181a-1mutant M3
aacauaaaacgcugucggugagu

SEQ ID NO: 79 - RNA, 23 nt, Artificial Sequence
  Chemically synthesized mir-181a-1mutant M4
aacauucuucgcugucggugagu

SEQ ID NO: 80 - RNA, 23 nt, Artificial Sequence
  Chemically synthesized mir-181a-1mutant M5
aacauucaaaccugucggugagu

SEQ ID NO: 81 - RNA, 23 nt, Artificial Sequence
  Chemically synthesized mir-181a-1mutant M6
aacauucaacgaagucggugagu

SEQ ID NO: 82 - RNA, 23 nt, Artificial Sequence
  Chemically synthesized mir-181a-1mutant M7
aacauucaacgcucacggugagu

SEQ ID NO: 83 - RNA, 23 nt, Artificial Sequence
  Chemically synthesized mir-181a-1mutant M8
aacauucaacgcuguacgugagu

SEQ ID NO: 84 - RNA, 23 nt, Artificial Sequence
  Chemically synthesized mir-181a-1mutant M9
aacauucaacgcugucgcagagu

SEQ ID NO: 85 - RNA, 23 nt, Artificial Sequence
  Chemically synthesized mir-181a-1mutant M10
aacauucaacgcugucggucugu

SEQ ID NO: 86 - RNA, 23 nt, Artificial Sequence
  Chemically synthesized mir-181a-1mutant M11
aacauucaacgcugucggugaca

SEQ ID NO: 87 - RNA, 23 nt, Artificial Sequence
  Chemically synthesized mir-181a-1mutant SM1
auauaaaaacgcugucggugagu

SEQ ID NO: 88 - RNA, 23 nt, Artificial Sequence
  Chemically synthesized mir-181a-1mutant SM2
aacauucuuacaacacggugagu

SEQ ID NO: 89 - RNA, 23 nt, Artificial Sequence
  Chemically synthesized mir-181a-1mutant SM3
aacauucaacgcuguaccacuca

SEQ ID NO: 90 - RNA, 23 nt, Artificial Sequence
  Chemically synthesized mir-181a-1mutant SM4
aacauucuuacaacaaccacuca

SEQ ID NO: 91 - RNA, 60 nt, Artificial Sequence
  Chemically synthesized mir-181a-1mutant M1
auaauucaacgcugucggugaguuuggaauucaaauaaaaaccaucgaccguugauuuaa

SEQ ID NO: 92 - RNA, 60 nt, Artificial Sequence
  Chemically synthesized mir-181a-1mutant M2
aacuaucaacgcugucggugaguuuggaauucaaauaaaaaccaucgaccguugaaagua

SEQ ID NO: 93 - RNA, 60 nt, Artificial Sequence
  Chemically synthesized mir-181a-1mutant M3
aacauaaaacgcugucggugaguuuggaauucaaauaaaaaccaucgaccguuuuuugua

SEQ ID NO: 94 - RNA, 60 nt, Artificial Sequence
  Chemically synthesized mir-181a-1mutant M4
aacauucuucgcugucggugaguuuggaauucaaauaaaaaccaucgaccgaagauugua

SEQ ID NO: 95 - RNA, 60 nt, Artificial Sequence
  Chemically synthesized mir-181a-1mutant M5
aacauucaaaccugucggugaguuuggaauucaaauaaaaaccaucgacguuugauugua

SEQ ID NO: 96 - RNA, 60 nt, Artificial Sequence
  Chemically synthesized mir-181a-1mutant M6
aacauucaacgaagucggugaguuuggaauucaaauaaaaaccaucgaccguugauugua

SEQ ID NO: 97 - RNA, 60 nt, Artificial Sequence
  Chemically synthesized mir-181a-1mutant M7
aacauucaacgcucacggugaguuuggaauucaaauaaaaaccaucgugcguugauugua

SEQ ID NO: 98 - RNA, 60 nt, Artificial Sequence
  Chemically synthesized mir-181a-1mutant M8
aacauucaacgcuguacgugaguuuggaauucaaauaaaaaccauguaccguugauugua

SEQ ID NO: 99 - RNA, 60 nt, Artificial Sequence
  Chemically synthesized mir-181a-1mutant M9
aacauucaacgcugucgcagaguuuggaauucaaauaaaaaccugcgaccguugauugua

SEQ ID NO: 100 - RNA, 60 nt, Artificial Sequence
  Chemically synthesized mir-181a-1mutant M10
aacauucaacgcugucggucuguuuggaauucaaauaaaaacgaucgaccguugauugua

SEQ ID NO: 101 - RNA, 60 nt, Artificial Sequence
  Chemically synthesized mir-181a-1mutant M11
aacauucaacgcugucggugacauuggaauucaaauaaaaugcaucgaccguugauugua

SEQ ID NO: 102 - RNA, 60 nt, Artificial Sequence
  Chemically synthesized mir-181a-1mutant SM1
auauaaaaacgcugucggugaguuuggaauucaaauaaaaaccaucgaccguuuuuauaa

SEQ ID NO: 103 - RNA, 60 nt, Artificial Sequence
  Chemically synthesized mir-181a-1mutant SM2
aacauucuuacaacacggugaguuuggaauucaaauaaaaaccaucgugguaagauugua

SEQ ID NO: 104 - RNA, 60 nt, Artificial Sequence
  Chemically synthesized mir-181a-1mutant SM3
aacauucaacgcuguaccacucauuggaauucaaauaaaauggugguaccguugauugua

SEQ ID NO: 105 - RNA, 60 nt, Artificial Sequence
  Chemically synthesized mir-181a-1mutant SM4
aacauucuuacaacaaccacucauuggaauucaaauaaaauggugguugguaagauugua

SEQ ID NO: 106 - RNA, 23 nt, Artificial Sequence
  Chemically synthesized sequence derived from Mouse miR-181a
aacauucaacgcugucggugagu

SEQ ID NO: 107 - RNA, 22 nt, Artificial Sequence
  Chemically synthesized sequence derived from Mouse miR-181b
aacauucauugcugucgguggg

SEQ ID NO: 108 - RNA, 22 nt, Artificial Sequence
  Chemically synthesized sequence derived from Mouse miR-181c
aacauucaaccugucggugagu

SEQ ID NO: 109 - RNA, 24 nt, Artificial Sequence
  Chemically synthesized sequence derived from Mouse miR-181d
aacauucauuguugucgguggguu

SEQ ID NO: 110 - RNA, 60 nt, Artificial Sequence; identical to SEQ ID NO: 62
  Chemically synthesized sequence derived from Mouse pre-mir-181a-1

SEQ ID NO: 111 - RNA, 64 nt, Artificial Sequence
  Chemically synthesized sequence derived from Mouse pre-mir-181a-2
aacauucaacgcugucggugaguuugggauucaaaaacaaaaaaaccaccgaccguugacugua

SEQ ID NO: 112 - RNA, 60 nt, Artificial Sequence
  Chemically synthesized sequence derived from Mouse pre-mir-181b-1
aacauucauugcugucgguggguugaacuguguagaaaagcucacugaacaaugaaugca

SEQ ID NO: 113 - RNA, 58 nt, Artificial Sequence
  Chemically synthesized sequence derived from Mouse pre-mir-181b-2
aacauucauugcugucgguggguuugaaugucaaccaacucacugaucaaugaaugca

SEQ ID NO: 114 - RNA, 59 nt, Artificial Sequence; identical to SEQ ID NO: 63
  Chemically synthesized sequence derived from Mouse pre-mir-181c

SEQ ID NO: 115 - RNA, 62 nt, Artificial Sequence
  Chemically synthesized sequence derived from Mouse pre-mir-181d
aacauucauuguugucgguggguugugaggaggcagccagacccaccgggggaugaauguca

SEQ ID NO: 116 - RNA, 60 nt, Artificial Sequence
  Chemically synthesized sequence derived from Human pre-mir-181a-1
aacauucaacgcugucggugaguuuggaauuaaaaucaaaaccaucgaccguugauugua

SEQ ID NO: 117 - RNA, 58 nt, Artificial Sequence
  Chemically synthesized sequence derived from Human pre-mir-181a-2
aacauucaacgcugucggugaguuugggauuugaaaaaaccacugaccguugacugua

SEQ ID NO: 118 - RNA, 60 nt, Artificial Sequence
  Chemically synthesized sequence derived from Human pre-mir-181b-1
aacauucauugcugucgguggguugaacuguguggacaagcucacugaacaaugaaugca

SEQ ID NO: 119 - RNA, 58 nt, Artificial Sequence
  Chemically synthesized sequence derived from Human pre-mir-181b-2
aacauucauugcugucgguggguuugagucugaaucaacucacugaucaaugaaugca

SEQ ID NO: 120 - RNA, 59 nt, Artificial Sequence
  Chemically synthesized sequence derived from Human pre-mir-181c
aacauucaaccugucggugaguuugggcagcucaggcaaaccaucgaccguugagugga

SEQ ID NO: 121 - RNA, 63 nt, Artificial Sequence
  Chemically synthesized sequence derived from Human pre-mir-181d
aacauucauuguugucgguggguugugaggacugaggccagacccaccgggggaugaauguca

SEQ ID NO: 122 - RNA, 17 nt, Artificial Sequence
  Chemically synthesized sequence, ppy-mir-181a-1, derived from Pongo pygmaeus
uuggaauuaaaaucaaa

SEQ ID NO: 123 - RNA, 17 nt, Artificial Sequence
  Chemically synthesized sequence, ptr-mir-181a-1, derived from Pan troglodytes
uuggaauuaaaaucaaa

SEQ ID NO: 124 - RNA, 17 nt, Artificial Sequence
  Chemically synthesized sequence, ppa-mir-181a-1, derived from Pan paniscus
uuggaauuaaaaucaaa

SEQ ID NO: 125 - RNA, 17 nt, Artificial Sequence
  Chemically synthesized sequence, mne-mir-181a-1, derived from Macaca nemestrina
uuggaauuaaaaucaaa

SEQ ID NO: 126 - RNA, 17 nt, Artificial Sequence
  Chemically synthesized sequence, mml-mir-181a-1, derived from Macaca mulatta
uuggaauuaaaaucaaa

SEQ ID NO: 127 - RNA, 17 nt, Artificial Sequence
  Chemically synthesized sequence, lla-mir-181a-1, derived from Lagothrix lagotricha
uuggaauuaaaaucaaa

SEQ ID NO: 128 - RNA, 17 nt, Artificial Sequence
  Chemically synthesized sequence, hsa-mir-181a-1, derived from Homo sapiens
uuggaauuaaaaucaaa

SEQ ID NO: 129 - RNA, 17 nt, Artificial Sequence
  Chemically synthesized sequence, ggo-mir-181a-1, derived from Gorilla gorilla
uuggaauuaaaaucaaa

SEQ ID NO: 130 - RNA, 17 nt, Artificial Sequence
  Chemically synthesized sequence, mmu-mir-181a-1, derived from Mus musculus
uuggaauucaaauaaaa

SEQ ID NO: 131 - RNA, 18 nt, Artificial Sequence
  Chemically synthesized sequence, rno-mir-181a-1, derived from Rattus norvegicus
uuggaauucaaauaaaaa

SEQ ID NO: 132 - RNA, 17 nt, Artificial Sequence
  Chemically synthesized sequence, gga-mir-181a-1, derived from Gallus gallus
uuggaauuuaagugaaa

SEQ ID NO: 133 - RNA, 18 nt, Artificial Sequence
  Chemically synthesized sequence, fru-mir-181a-1, derived from Fugu rubripes
uugagcuaaauuggaaaa

SEQ ID NO: 134 - RNA, 18 nt, Artificial Sequence
  Chemically synthesized sequence, tni-mir-181a-1, derived from Tetraodon nigroviridis
uugagccaaauuggaaaa

SEQ ID NO: 135 - RNA, 18 nt, Artificial Sequence
  Chemically synthesized sequence, dre-mir-181a-1, derived from Danio rerio
uugagcuaaauggaaaaa

SEQ ID NO: 136 - RNA, 17 nt, Artificial Sequence
  Chemically synthesized sequence, ggo-mir-181c, derived from Gorilla gorilla
uugggcagcucaggcaa

SEQ ID NO: 137 - RNA, 17 nt, Artificial Sequence
  Chemically synthesized sequence, hsa-mir-181c, derived from Homo sapiens
uugggcagcucaggcaa

SEQ ID NO: 138 - RNA, 17 nt, Artificial Sequence
  Chemically synthesized sequence, mml-mir-181c, derived from Macaca mulatta
uugggcagcucaggcaa

SEQ ID NO: 139 - RNA, 17 nt, Artificial Sequence
  Chemically synthesized sequence, ppa-mir-181c, derived from Pan paniscus
uugggcagcucaggcaa

SEQ ID NO: 140 - RNA, 17 nt, Artificial Sequence
  Chemically synthesized sequence, ptr-mir-181c, derived from Pan troglodytes
uugggcagcucaggcaa

SEQ ID NO: 141 - RNA, 17 nt, Artificial Sequence
  Chemically synthesized sequence, ssc-mir-181c, derived from Sus scrofa
uugggcagcucaggcaa

SEQ ID NO: 142 - RNA, 17 nt, Artificial Sequence
  Chemically synthesized sequence, mmu-mir-181c, derived from Mus musculus
uugggcagcucagacaa

SEQ ID NO: 143 - RNA, 17 nt, Artificial Sequence
  Chemically synthesized sequence, rno-mir-181c, derived from Rattus norvegicus
uugggcagcucagacaa